(19) United States
(12) Patent Application Publication
(10) Pub. No.: US 2025/0195553 A1
(43) Pub. Date: Jun. 19, 2025

(54) Use of Genipin-1-B-D-Gentiobioside in Preparation of Drug

(71) Applicant: Guizhou Bailing Group Pharmaceutical Co., Ltd., Anshun (CN)

(72) Inventors: Xiaolan Cui, Anshun (CN); Chenggang Huang, Anshun (CN); Wen Xia, Anshun (CN); Yujie Pan, Anshun (CN); Guangyuan Qin, Anshun (CN); Xing Li, Anshun (CN); Zhou Xu, Anshun (CN); Lei Bao, Anshun (CN); Yunqin Chen, Anshun (CN); Shenglong Yan, Anshun (CN); Kun Jiang, Anshun (CN)

(21) Appl. No.: 19/026,598

(22) Filed: Jan. 17, 2025

Related U.S. Application Data
(63) Continuation of application No. PCT/CN2023/138805, filed on Dec. 14, 2023.

(51) Int. Cl. A61K 31/7048; A61K 9/00; A61K 36/744; A61P 31/14

(52) U.S. Cl. CPC A61K 31/7048; A61K 9/0019; A61K 9/0078; A61K 36/744; A61P 31/14; A61K 2236/15; A61K 2236/39; A61K 2236/53

(57) ABSTRACT

The present disclosure relates to a use of Genipin-1-β-D-gentiobioside in preparation of a drug, specifically for use in a drug that treats respiratory inflammatory injury caused by coronavirus, respiratory syncytial virus, or Mycoplasma pneumoniae infection. The present disclosure has discovered that Genipin-1-β-D-gentiobioside possesses the characteristics of reducing viral load, lung index, and inflammatory cytokines in lung tissue following coronavirus infection, respiratory syncytial virus infection, or Mycoplasma pneumoniae infection. It also demonstrates a significant protective effect against death caused by coronavirus infection.

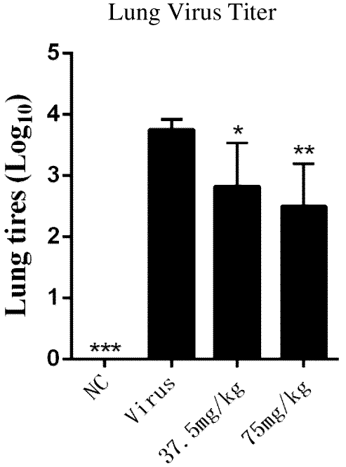

### CROSS-REFERENCE TO RELATED APPLICATIONS

This application is a continuation of International Application No. PCT/CN2023/138805, filed on Dec. 14, 2023. All of the aforementioned applications are incorporated herein by reference in their entireties.

### TECHNICAL FIELD

The present disclosure relates to a use of Genipin-1-β-D-gentiobioside, in particular to a use of Genipin-1-β-D-gentiobioside in preparation of a drug for treating coronavirus infection, respiratory syncytial virus infection or Mycoplasma pneumoniae infection.

### BACKGROUND

Coronaviruses represent the largest known group of RNA viruses, characterized as enveloped viruses with a positive-sense RNA genome. They possess a wide range of natural hosts and are significant pathogens for both humans and vertebrates. Coronaviruses can infect the respiratory tract, gastrointestinal tract, liver, kidneys, and nervous system of humans, leading to life-threatening conditions such as pneumonia and bronchitis. Among coronaviruses (CoV), the envelope glycoprotein spike (S) is responsible for viral entry into cells and intercellular transmission. Upon entering cells via endocytosis, coronaviruses release their viral RNA and nucleocapsid into the cytoplasm, utilizing the host's machinery for replication and completing the viral replication cycle. Coronaviruses can cause acute respiratory infections in humans, with the notable example of the new coronavirus causing acute pneumonia and, in severe cases, death.

Currently, drugs for treating coronavirus infections primarily consist of small-molecule compounds that target the viral replication cycle, such as Paxilovid, Molnupiravir, Azvudine and VV116. Due to their single targets and limited efficacy, these drugs struggle to address viral mutations and drug resistance, particularly in severe or fatal cases resulting from factors such as inflammatory cytokine storms, disseminated intravascular coagulation, and impaired immune function.

Respiratory syncytial virus (RSV) is a negative-sense single-stranded RNAvirus belonging to the Pneumoviridae family. Human RSV is a highly contagious virus that commonly causes acute lower respiratory tract infections in infants and contributes to high mortality rates in both the elderly and children. RSV can trigger respiratory infections ranging from mild to severe, including pneumonia and bronchitis, and may lead to serious complications such as respiratory failure. In current medical practice, ribavirin is the preferred anti-RSV drug for RSV pneumonia; however, due to its multiple side effects, it is rarely used clinically. Therefore, the treatment of RSV pneumonia currently relies on comprehensive therapy (combining anti-infective agents, glucocorticoids, bronchoscopy, anticoagulants, etc.), and there is still a lack of effective drugs.

Mycoplasma pneumoniae (MP) is an important pathogenic microorganism causing respiratory infections in children. Mycoplasma pneumoniae pneumonia (MPP) is currently the most common community-acquired pneumonia (CAP) among children aged 5 and above in China. Previous data indicate that during epidemics, MPP accounts for 20%-40% of CAP cases in the general population and up to 70% in closed populations. While MPP was previously considered to have mild or even self-limiting clinical manifestations, an increasing number of severe MPP (SMPP) cases have been reported in recent years.

In current medical practice, macrolide antibiotics are the first-choice treatment for MPP; however, the emergence of drug resistance in recent years has resulted in reduced efficacy. New tetracycline antibiotics, such as doxycycline and minocycline, are alternative treatments for MPP and have demonstrated efficacy against drug-resistant MPP. Nonetheless, they may cause side effects such as tooth discoloration and enamel dysplasia, limiting their clinical application. Quinolone antibiotics are also alternative treatments for MPP, particularly effective against macrolide-resistant MPP, and are used in the treatment of suspected or confirmed MP-resistant MUMPP, RMMP, and SMPP. However, quinolones carry the risk of cartilage damage in young animals and tendon rupture in humans, thus their clinical use is restricted. In summary, the current treatment for MPP primarily relies on comprehensive therapy (combining anti-infective agents, glucocorticoids, bronchoscopy, anticoagulants, etc.), and there is still a lack of effective drugs.

### SUMMARY

The objective of the present disclosure is to provide a use of Genipin-1-β-D-gentiobioside in preparation of a drug. The present disclosure has discovered that Genipin-1-β-D-gentiobioside possesses the characteristics of reducing viral load, lung index, and inflammatory cytokines in lung tissue following coronavirus infection, respiratory syncytial virus infection, or Mycoplasma pneumoniae infection. It also demonstrates a significant protective effect against death caused by coronavirus infection.

Technical solution of the present disclosure: a use of Genipin-1-β-D-gentiobioside in preparation of a drug for treating respiratory injury caused by coronavirus, respiratory syncytial virus or Mycoplasma pneumoniae infections.

According to the aforementioned use of Genipin-1-β-D-gentiobioside in preparation of a drug, the Genipin-1-β-D-gentiobioside is used in preparation of a drug for treating pneumonia inflammatory injury caused by a coronavirus, respiratory syncytial virus or Mycoplasma pneumoniae infection.

According to the aforementioned use of the Genipin-1-β-D-gentiobioside in preparation of the drug, the Genipin-1-β-D-gentiobioside is used in preparation of a drug for treating coronavirus, respiratory syncytial virus or Mycoplasma pneumoniae infections, which further exhibits a protective effect against mortality.

According to the aforementioned use of the Genipin-1-β-D-gentiobioside in preparation of the drug, the coronavirus comprises any one of Severe Acute Respiratory Syndrome Coronavirus 2 (SARS-CoV-2), Human Coronavirus 229E (HCoV-229E) and Human Coronavirus OC43 (HCoV-OC43).

According to the aforementioned use of the Genipin-1-β-D-gentiobioside in preparation of the drug, administration routes of the Genipin-1-β-D-gentiobioside comprise any one of inhalation, oral administration, injection, sublingual administration, spraying or rectal administration; and dosage forms of the drug comprise any one of inhalation, oral, injection, spray, film and suppository.

A nebulized inhalation solution containing a Genipin-1-β-D-gentiobioside, realizing the aforementioned use, wherein the nebulized inhalation solution comprises following ingredients: the Genipin-1-β-D-gentiobioside, a pH regulator that adjusts pH to 4.5˜7.0, an osmotic pressure regulator that accounts for 0˜0.9% of a weight of the Genipin-1-β-D-gentiobioside and a solvent.

According to the aforementioned nebulized inhalation solution, a preparation method of the nebulized inhalation solution comprises following steps:
(1) measuring 40%˜90% (v/v) of total solution volume required for preparation using water for injection to obtain a first solution;
(2) adding the osmotic pressure regulator into the first solution at a temperature of 20˜80° C., and stirring uniformly to obtain a second solution;
(3) adding the pH regulator and the Genipin-1-β-D-gentiobioside into the second solution, and adjusting a pH of the second solution to a target value to obtain a third solution; and
(4) adding solvent into the third solution to reach total solution volume required for preparation, and stirring uniformly to obtain the nebulized inhalation solution containing the Genipin-1-β-D-gentiobioside.

An injection containing a Genipin-1-β-D-gentiobioside, realizing the aforementioned use, wherein the injection comprises following ingredients: the Genipin-1-β-D-gentiobioside, a pH regulator that adjusts pH to 4.5-7.0, an osmotic pressure regulator that adjusts the osmolarity to isotonicity and an injection solvent.

According to the aforementioned injection, a preparation method of the injection comprises following steps:
(1) measuring 40%˜90% (v/v) of total solution volume required for preparation using water for injection to obtain a first solution;
(2) adding the osmotic pressure regulator into the first solution at a temperature of 20˜80° C., and stirring uniformly to obtain a second solution;
(3) adding the pH regulator and the Genipin-1-β-D-gentiobioside into the second solution, and adjusting a pH of the second solution to a target value to obtain a third solution; and
(4) adding solvent into the third solution to reach total solution volume required for preparation, and stirring uniformly to obtain the injection containing the Genipin-1-β-D-gentiobioside.

A preparation method of a Genipin-1-β-D-gentiobioside, used for extracting the Genipin-1-β-D-gentiobioside in aforementioned uses, wherein the method comprises following operation steps:
a, taking Gardeniae Fructus, performing water extraction to obtain an extract, concentrating the extract under reduced pressure to achieve a crude drug content of 0.03˜0.2 g/ml in the extract;
b, loading the extract onto a macroporous resin column, eluting first with 1˜5 times column volumes of deionized water, then eluting with 1˜5 times column volumes of 10˜20% ethanol, collecting ethanol eluate, recovering ethanol under reduced pressure, concentrating to a volume that is 0.1 times the original volume of the crude drug extract, adding ethanol to achieve a 90% ethanol concentration, letting the solution stand, allowing precipitation, filtering, passing supernatant obtained from ethanol precipitation through a neutral alumina column, eluting sequentially with 1˜8 times column volumes of 50˜90% ethanol, collecting ethanol eluate obtained with 50˜60% ethanol, recovering ethanol under reduced pressure, and drying to obtain a crude product; and
c. refining the crude product by hot ethanol dissolution and recrystallization for 2˜3 times to obtain a refined product, drying the refined product, and removing ethanol to obtain high-purity Genipin-1-β-D-gentiobioside.

The term “crude drug content” refers to the ratio of the weight of the medicinal material input (g) to the volume of the medicinal solution (mL). For instance, if 2 kg of Gardeniae Fructus (a medicinal herb) is decocted, filtered, and then concentrated to 60 L, the resulting crude drug concentration would be 2 kg/60 L=0.033 g/mL.

The phrase “concentrating to a volume that is 0.1 times the original volume of the crude drug extract” means reducing the volume of the extract by removing the solvent, so that the final volume is 10% (i.e., 0.1 times) of the original volume of the crude drug.

According to the aforementioned preparation method of the Genipin-1-β-D-gentiobioside, in step a, the specific steps for water extraction are: crushing Gardeniae Fructus, boiling crused Gardeniae Fructus with 12 times, 10 times and 10 times the amount of water respectively for 1˜1.5 hours each time; and in step b, a wet volume ratio of macroporous resin to a weight of Gardeniae Fructus is 3:2˜3 ml/g, and a weight ratio of neutral alumina to the weight of Gardeniae Fructus is 1:3˜3.5.

According to the aforementioned preparation method of the Genipin-1-β-D-gentiobioside, step b specifically involves loading Gardeniae Fructus extract onto an NKA-9 macroporous resin column, eluting first with 2 times column volumes of deionized water, collecting sample solution and water eluate, combining the sample solution and the water eluate, and then loading the combined solution onto an X-5 macroporous resin column, eluting first with 1 times column volume of deionized water, then eluting with 5 times column volumes of 10% ethanol, collecting ethanol eluate, recovering ethanol under reduced pressure, concentrating to a relative density of 1.08˜1.15 at 60° C., adding ethanol to achieve a 90% ethanol concentration, letting the solution stand, allowing precipitation, filtering, passing supernatant obtained from ethanol precipitation through the neutral alumina column, eluting sequentially with 6 times column volumes of 90% ethanol and 4 times column volumes of 60% ethanol, collecting eluate obtained with 60% ethanol, recovering ethanol under reduced pressure, and drying to obtain the crude product.

According to the aforementioned preparation method of the Genipin-1-β-D-gentiobioside, in step c, the specific steps for recrystallization after hot ethanol dissolution of the crude product are: adding the crude product to 0.5˜1 times the amount of anhydrous ethanol, heating and refluxing to dissolve the crude product, filtering while hot, letting the solution stand, allowing crystallization, and filtering by suction to obtain the refined product.

Compared with the prior art, the beneficial effects of the present disclosure are as follows:

Through animal experiments, the present disclosure has discovered and demonstrated that Genipin-1-β-D-gentiobioside exhibits a significant protective effect against mouse deaths caused by SARS-CoV-2 infection. Genipin-1-β-D-gentiobioside demonstrates high inhibition rates on coronavirus infection, respiratory syncytial virus (RSV) infection, and lung index in Mycoplasma pneumoniae-infected mice. Furthermore, Genipin-1-β-D-gentiobioside reduces the viral load in mice infected with coronavirus, RSV, or Mycoplasma pneumoniae, and decreases the levels of inflammatory cytokines TNF-α, IL-6, and IL-10 in the lung tissues of these infected mice.

Consequently, Genipin-1-β-D-gentiobioside, by reducing various inflammatory cytokines and inhibiting cytokine storms following coronavirus, RSV, or Mycoplasma pneumoniae infections, significantly lowers mortality rates, lung indices, and viral loads in lung tissues. Genipin-1-β-D-gentiobioside exhibits a definite and remarkable therapeutic effect on viral pneumonia caused by coronavirus, RSV, or Mycoplasma pneumoniae infections. These findings indicate the anti-coronavirus, anti-RSV, and anti-Mycoplasma pneumoniae activities of Genipin-1-β-D-gentiobioside, suggesting its broad-spectrum nature. Genipin-1-β-D-gentiobioside can be used to treat respiratory diseases and severe pneumonia caused by coronavirus, RSV, and Mycoplasma pneumoniae infections, providing a protective effect against inflammatory damage and death. Therefore, Genipin-1-β-D-gentiobioside has potential applications in the preparation of drugs for the treatment of respiratory diseases, inflammatory lung injuries, and those offering protection against pneumonia-related deaths caused by coronavirus, RSV, or Mycoplasma pneumoniae infections.

The administration routes of Genipin-1-β-D-gentiobioside include inhalation, oral administration, injection, sublingual administration, spraying and rectal administration. Genipin-1-β-D-gentiobioside can be formulated into various common pharmaceutical preparations in medical and pharmaceutical studies.

The present disclosure also provides a method for preparing Genipin-1-β-D-gentiobioside. The crude product obtained by this method has removed most of the pigments and iridoid impurities, with a Genipin-1-β-D-gentiobioside content exceeding 60%. This method enables efficient separation to obtain the active ingredient that meets the requirements for new drugs, where the content of the active ingredient (Genipin-1-β-D-gentiobioside) reaches over 50% of the extract. In the final finished extract, the purity of Genipin-1-β-D-gentiobioside exceeds 96%.

Moreover, during the preparation process of the present disclosure, toxic and harmful reagents are avoided, and reusable macroporous resins are selected for purification. Therefore, the process route of this method is green, safe, and relatively low in cost, making it suitable for large-scale production.

### BRIEF DESCRIPTION OF THE DRAWINGS

The sole FIGURE is a graph showing the viral titer detection in the lung tissues of mice infected with new coronavirus.

### DETAILED DESCRIPTION OF THE EMBODIMENTS

The present disclosure is further described below with reference to the embodiments, but is not intended to limit the present disclosure.

The present disclosure discovers a use of Genipin-1-β-D-gentiobioside in preparation of a drug for treating coronavirus, respiratory syncytial virus or Mycoplasma pneumoniae infections.

The Genipin-1-β-D-gentiobioside is used in preparation of a drug for treating respiratory injury caused by coronavirus, respiratory syncytial virus or Mycoplasma pneumoniae infections.

The Genipin-1-β-D-gentiobioside is used in preparation of a drug for treating pneumonia inflammatory injury caused by coronavirus, respiratory syncytial virus or Mycoplasma pneumoniae infections.

The Genipin-1-β-D-gentiobioside is used in preparation of a drug for treating coronavirus, respiratory syncytial virus or Mycoplasma pneumoniae infections, which further exhibits a protective effect against mortality.

Furthermore, the Genipin-1-β-D-gentiobioside is used in preparation of a drug for treating increased levels of inflammatory cytokines TNF-α, IL-6 and IL-10 in lung tissue cells caused by coronavirus, respiratory syncytial virus or Mycoplasma pneumoniae infections.

The coronavirus comprises any one of Severe Acute Respiratory Syndrome Coronavirus 2 (SARS-CoV-2), Human Coronavirus 229E (HCoV-229E) and Human Coronavirus OC43 (HCoV-OC43).

The Genipin-1-β-D-gentiobioside in the present disclosure can be extracted from the traditional Chinese medicine Gardeniae Fructus or prepared through synthetic methods.

The method for extracting Genipin-1-β-D-gentiobioside from Gardeniae Fructus involves the following operational steps:
a. extracting the medicinal material: Crushing the Gardeniae Fructus, decocting the crushed Gardeniae Fructus three times with 12, 10, and 10 times the amount of water respectively for 1 to 1.5 hours each time, and then concentrating the extract under reduced pressure to achieve a crude drug content of 0.03 to 0.2 g/ml;
b. preparing the crude product: Loading the extract onto a macroporous resin column, preferably NKA-9 or X-5, with a wet volume to Gardeniae Fructus weight ratio of 3:2 to 3 ml/g. Eluting the column first with 1 to 5 times the column volume of deionized water, then with 1 to 5 times the column volume of 10 to 20% ethanol. Collecting the ethanol eluate, recovering the ethanol under reduced pressure, concentrating the ethanol eluate to 0.1 times the original volume of the crude drug extract, adjusting the ethanol concentration to 90%, letting the solution stand, allowing precipitation, filtering, passing the supernatant through a neutral alumina column (100 to 200 mesh, with a weight ratio to Gardeniae Fructus of 1:3 to 3.5), eluting sequentially with 1 to 8 times the column volumes of 50 to 90% ethanol, collecting the eluate obtained with 50 to 60% ethanol, recovering the ethanol under reduced pressure, and finally drying to obtain the crude product; and
c. preparing Genipin-1-β-D-gentiobioside: Refining the crude product through hot ethanol dissolution and recrystallization for 2 to 3 times. The refinement process involves dissolving the crude product into 0.5 to 1 times its weight of anhydrous ethanol by heating and refluxing, filtering while hot, letting the solution stand, allowing crystallization, filtering by suction to obtain the refined product, drying the refined product, and then removing the ethanol to obtain high-purity Genipin-1-β-D-gentiobioside.

The administration routes of Genipin-1-β-D-gentiobioside comprise any one of inhalation, oral administration, injection, sublingual administration, spraying, or rectal administration.

The present disclosure employs inhalation and injection as the routes of administration, targeting the respiratory tract and lungs. The drug is administered directly in a nebulized form to the respiratory tract and lungs, offering advantages such as rapid onset of action, high local drug concentration at the inflammatory sites in the respiratory tract and lungs, low dosage, ease of application, and reduced systemic adverse reactions. This makes it an important therapeutic approach for respiratory diseases.

The present disclosure also provides a drug for treating coronavirus infections, comprising Genipin-1-β-D-gentiobioside and pharmaceutically acceptable excipients.

The dosage forms of the drug comprise any one of inhalation, oral, injection, spray, film and suppository.

Administration through inhalants, injectables, sprays, suppositories, and films (non-oral films) can avoid the first-pass effect in the liver and intestine, preventing the degradation and inactivation of Genipin-1-β-D-gentiobioside, which contains glycosidic bonds in its structure, by gastric acid and intestinal bacteria.

An inhalation formulation containing Genipin-1-β-D-gentiobioside, used for the aforementioned application, comprises the following ingredients: 20 to 200 mg of Genipin-1-β-D-gentiobioside, a pH adjuster that adjusts pH to 4.5 to 7.0, an osmotic pressure regulator that accounts for 0˜0.9% of a weight of the Genipin-1-β-D-gentiobioside, and 1 to 5 ml of a solvent.

The preparation method of the inhalation formulation containing Genipin-1-β-D-gentiobioside comprises the following steps:
(1) measuring 40%˜90% (v/v) of total solution volume required for preparation using water for injection to obtain a first solution;
(2) adding the osmotic pressure regulator into the first solution at a temperature of 20˜80° C., and stirring uniformly to obtain a second solution;
(3) adding the pH regulator and the Genipin-1-β-D-gentiobioside into the second solution, and adjusting a pH of the second solution to a target value to obtain a third solution; and
(4) adding solvent into the third solution to reach total solution volume required for preparation, stirring uniformly, and filtering through a 0.22 μm filter membrane or filter element to obtain the nebulized inhalation solution containing the Genipin-1-β-D-gentiobioside.

An injection containing Genipin-1-β-D-gentiobioside, used for the aforementioned application, comprises the following ingredients: 20 to 200 mg of Genipin-1-β-D-gentiobioside, a pH adjuster that adjusts pH to 4.5 to 7.0, an osmotic pressure regulator that adjusts the osmolarity to isotonicity, and 1 to 5 ml of an injection solvent.

The preparation method of the injection comprises following steps:
(1) measuring 40%˜90% (v/v) of total solution volume required for preparation using water for injection to obtain a first solution;
(2) adding the osmotic pressure regulator into the first solution at a temperature of 20˜80° C., and stirring uniformly to obtain a second solution;
(3) adding the pH regulator and the Genipin-1-β-D-gentiobioside into the second solution, and adjusting a pH of the second solution to a target value to obtain a third solution; and
(4) adding solvent into the third solution to reach total solution volume required for preparation, and stirring uniformly to obtain the injection containing the Genipin-1-β-D-gentiobioside.

### Extraction Embodiment 1

The preparation method of Genipin-1-β-D-gentiobioside comprises following operation steps:
a. extracting the medicinal material: Taking 2 kg of Gardeniae Fructus, crushing the Gardeniae Fructus, decocting the crushed Gardeniae Fructus three times with 12, 10, and 10 times the amount of water respectively for 1 hour each time, combining and filtering the extract, and then concentrating the extract under reduced pressure to achieve a crude drug content of 0.033 g/ml;
b. preparing the crude product: Loading Gardeniae Fructus extract onto an NKA-9 macroporous resin column, eluting first with 2 times column volumes of deionized water, collecting sample solution and water eluate, combining the sample solution and the water eluate, and then loading the combined solution onto an X-5 macroporous resin column, eluting first with 1 times column volume of deionized water, then eluting with 5 times column volumes of 10% ethanol, collecting ethanol eluate, recovering ethanol under reduced pressure, concentrating the ethanol eluate to 200 ml with a relative density of 1.15 at 60° C., adding ethanol to achieve a 90% ethanol concentration, letting the solution stand, allowing precipitation, filtering, passing supernatant obtained from ethanol precipitation through a 100-mesh neutral alumina column, eluting sequentially with 6 times column volumes of 90% ethanol and 4 times column volumes of 60% ethanol, collecting eluate obtained with 60% ethanol, recovering ethanol under reduced pressure, and drying to obtain the crude product; and
c. preparing Genipin-1-β-D-gentiobioside: Dissolving the crude product into 1.2 L of anhydrous ethanol by heating and refluxing, filtering while hot, letting the solution stand, allowing crystallization, filtering by suction to obtain Refined Product-1; dissolving Refined Product-1 into 1.4 L of anhydrous ethanol by heating and refluxing, filtering while hot, letting the solution stand, allowing crystallization, filtering by suction, removing the ethanol to obtain Refined Product-2. After drying Refined Product-2 (at 60° C., 0.7 MPa) and removing the ethanol, 11.7 g of high-purity Genipin-1-β-D-gentiobioside with a mass content of 96.2% is obtained.

### Extraction Embodiment 2

The preparation method for Genipin-1-β-D-gentiobioside comprises following operation steps:
a. extracting the medicinal material: Taking 5 kg of Gardeniae Fructus, crushing the Gardeniae Fructus, and performing water extraction. For the first extraction, add 12 times the volume of water and extract for 1.5 hours; for the second extraction, add 10 times the volume of water and extract for 1 hour; for the third extraction, add 10 times the volume of water again and extract for 1 hour. Combining and filtering the extract, and then concentrating the extract under reduced pressure to achieve a crude drug content of 0.2 g/ml;
b. preparing the crude product: Loading Gardeniae Fructus extract onto an NKA-9 macroporous resin column, eluting first with 3 times column volumes of deionized water, collecting sample solution and water eluate, combining the sample solution and the water eluate, and then loading the combined solution onto an X-5 macroporous resin column, eluting first with 1 times column volume of deionized water, then eluting with 3 times column volumes of 20% ethanol, collecting ethanol eluate, recovering ethanol under reduced pressure, concentrating the ethanol eluate to 1 L with a relative density of 1.08 at 60° C., adding ethanol to achieve a 90% ethanol concentration, letting the solution stand, allowing precipitation, filtering, passing supernatant obtained from ethanol precipitation through a 100-mesh neutral alumina column, eluting sequentially with 6 times column volumes of 90% ethanol and 8 times column volumes of 50% ethanol, collecting eluate obtained with 50% ethanol, recovering ethanol under reduced pressure, and drying to obtain the crude product; and
c. preparing Genipin-1-β-D-gentiobioside: Dissolving the crude product into 2.5 L of anhydrous ethanol by heating and refluxing, filtering while hot, letting the solution stand, allowing crystallization, filtering by suction to obtain Refined Product-1; dissolving Refined Product-1 into 2.5 L of anhydrous ethanol by heating and refluxing, filtering while hot, letting the solution stand, allowing crystallization, filtering by suction, removing the ethanol to obtain Refined Product-2; dissolving Refined Product-2 into 2.8 L of anhydrous ethanol by heating and refluxing, filtering while hot, letting the solution stand, allowing crystallization, filtering by suction, removing the ethanol to obtain Refined Product-3. After drying Refined Product-3 (at 60° C., 0.7 MPa) and removing the ethanol, 20.7 g of high-purity Genipin-1-β-D-gentiobioside with a mass content of 96.7% is obtained.

### Formulation Embodiment 1: Inhalation Administration—Inhalation Solution

An inhalation solution containing Genipin-1-β-D-gentiobioside has the following formulation: 20 mg of Genipin-1-β-D-gentiobioside, citric acid to adjust the pH to 4.5, sodium chloride to adjust the osmolarity to isotonicity, and solvent to a total volume of 1 ml.

The preparation method of the inhalation solution comprises the following steps:
(1) measuring 40% (v/v) of total solution volume required for preparation using water for injection to obtain a first solution;
(2) adding sodium chloride into the first solution at a controlled temperature of 25° C. and stirring until completely dissolved to obtain a second solution;
(3) adjusting the pH of the second solution to 4.5 with citric acid to obtain a third solution;
(4) adding Genipin-1-β-D-gentiobioside into the third solution, stirring uniformly, and supplementing with additional citric acid as needed to maintain the pH at 4.5 to obtain a fourth solution;
(5) adding solvent into the fourth solution to adjust the volume to the total required volume for the preparation, and stirring uniformly to obtain a fifth solution;
(6) filtering the fifth solution through a 0.22 μm filter membrane or filter cartridge to obtain a sixth solution; and
(7) filling the sixth solution into 1 ml-sized ampoules or vials, and sealing the ampoules or vials to obtain the inhalation solution of Genipin-1-β-D-gentiobioside.

### Formulation Embodiment 2: Inhalation Administration—Inhalation Solution

An inhalation solution containing Genipin-1-β-D-gentiobioside has the following formulation: 100 mg of Genipin-1-β-D-gentiobioside, 0.1% (w/v) citric acid, sodium citrate to adjust the pH to 5.0, and solvent to a total volume of 3 ml.

The preparation method of the aforementioned inhalation solution comprises the following steps:
(1) measuring 60% (v/v) of total solution volume required for preparation using water for injection to obtain a first solution;
(2) adding the citric acid into the first solution at a temperature of 50° C., and stirring uniformly to obtain a second solution;
(3) adjusting the pH of the second solution to 5.0 with sodium citrate to obtain a third solution;
(4) adding Genipin-1-β-D-gentiobioside into the third solution, stirring uniformly, and supplementing with additional sodium citrate as needed to maintain the pH at 5.0 to obtain a fourth solution;
(5) adding solvent into the fourth solution to adjust the volume to the total required volume for the preparation, stirring uniformly to obtain a fifth solution;
(6) filtering the fifth solution through a 0.22 μm filter membrane or filter cartridge to obtain a sixth solution; and
(7) filling the sixth solution into 3 ml-sized ampoules or vials, and sealing the ampoules or vials to obtain the inhalation solution of Genipin-1-β-D-gentiobioside.

### Formulation Embodiment 3: Inhalation Administration—Inhalation Solution

An inhalation solution containing Genipin-1-β-D-gentiobioside has the following formulation: 200 mg of Genipin-1-β-D-gentiobioside, 0.5% (w/v) sodium citrate, sodium chloride to adjust the osmolarity to isotonicity, sodium hydroxide to adjust the pH to 7.0, and solvent to a total volume of 5 ml.

The preparation method for the aforementioned inhalation solution comprises the following steps:
(1) measuring 90% (v/v) of total solution volume required for preparation using water for injection to obtain a first solution;
(2) adding the sodium citrate into the first solution at a temperature of 80° C., and stirring uniformly to obtain a second solution;
(3) adding sodium chloride into the second solution, and stirring until completely dissolved to obtain a third solution;
(4) adjusting the pH of the third solution to 7.0 with sodium hydroxide to obtain a fourth solution;
(5) adding Genipin-1-β-D-gentiobioside into the fourth solution, stirring uniformly, and supplementing with additional sodium hydroxide as needed to maintain the pH at 7.0 to obtain a fifth solution;
(6) adding solvent into the fifth solution to adjust the volume to the total required volume for the preparation, and stirring uniformly to obtain a sixth solution;
(7) filtering the sixth solution through a 0.22 μm filter membrane or filter cartridge to obtain a seventh solution; and
(8) filling the seventh solution into 5 ml-sized ampoules or vials, and sealing the ampoules or vials to obtain the inhalation solution of Genipin-1-β-D-gentiobioside.

For the Formulation Embodiments 1-3, when using, place the nebulizer (which can operate based on principles such as air compression, vibrating mesh, or ultrasonics) on a flat surface. During operation, keep the device away from textiles to avoid blocking the air inlet with textile fibers. After correctly installing the nebulization cup according to the user manual, open the medicine packaging box, take out the ampoule or vial, draw the liquid medicine into a syringe, and transfer it to the nebulization cup. Sit or stand upright to ensure normal breathing. After confirming that the nebulization mask covers your nose and mouth or the nebulization mouthpiece is placed in your mouth, press the nebulization button to start the nebulization process and continue inhaling until no more droplets are emitted. To reduce the risk of infection, illness, or contamination, clean and disinfect the nebulizer according to the instructions after treatment.

Stability testing experiments have shown that the quality and nebulization characteristics of the three Formulation Embodiments all meet the requirements when stored at 25° C. for six months.

### Formulation Embodiment 4: Inhalation Administration—Lyophilized Powder for Inhalation

A lyophilized powder for inhalation containing Genipin-1-β-D-gentiobioside has the following formulation: 20 mg of Genipin-1-β-D-gentiobioside, hydrochloric acid to adjust the pH to 4.5, and solvent to a total volume of 1 ml.

The preparation method of the aforementioned lyophilized powder for inhalation comprises the following steps:
(1) measuring 70% (v/v) of total solution volume required for preparation using water for injection to obtain a first solution;
(2) adding the sodium hydroxide into the first solution at a temperature of 30° C., adjusting the pH to 4.5, and stirring uniformly to obtain a second solution;
(3) adding Genipin-1-β-D-gentiobioside into the second solution, and stirring uniformly to obtain a third solution;
(4) measuring the pH, and if the pH is not within the range of 4.4 to 4.6, adding an appropriate amount of sodium hydroxide again to adjust the solution's pH to 4.5 to obtain a fourth solution;
(5) adding solvent into the fourth solution to adjust the volume to the total required volume for the preparation, and stirring uniformly to obtain a fifth solution;
(6) filtering the fifth solution through a 0.22 μm filter membrane or filter cartridge to obtain a sixth solution;
(7) filling the sixth solution into 10 ml-sized vials and applying half-stoppers; and
(8) transferring the aforementioned samples into the freeze-dryer, and lyophilizing according to the predetermined lyophilization curve: pre-freezing at −45° C. for 6 hours, followed by vacuum-pumping and heating to −15° C. for sublimation drying for 10 hours, then heating to 25° C. for desorption drying for 4 hours. Once lyophilization is finished, fully stoppering the vials, removing the vials from the freeze-dryer, crimping the caps, and obtaining lyophilized powder for inhalation containing Genipin-1-β-D-gentiobioside.

### Formulation Embodiment 5: Inhalation Administration—Lyophilized Powder for Inhalation

A lyophilized powder for inhalation containing Genipin-1-β-D-gentiobioside has the following formulation: 80 mg of Genipin-1-β-D-gentiobioside, 0.1% (w/v) citric acid, pH adjusted to 5.0 with an appropriate amount of sodium citrate, and solvent to a total volume of 2 ml.

The preparation method of the aforementioned lyophilized powder for inhalation comprises the following steps:
(1) measuring 50% (v/v) of total solution volume required for preparation using water for injection to obtain a first solution;
(2) adding the citric acid and sodium citrate into the first solution at a temperature of 40° C., adjusting the pH to 5.0, and stirring uniformly to obtain a second solution;
(3) adding Genipin-1-β-D-gentiobioside into the second solution, and stirring uniformly to obtain a third solution;
(4) measuring the pH, and if the pH is not within the range of 4.9 to 5.1, adding an appropriate amount of sodium citrate again to adjust the solution's pH to 5.0 to obtain a fourth solution;
(5) adding solvent into the fourth solution to adjust the volume to the total required volume for the preparation, and stirring uniformly to obtain a fifth solution;
(6) filtering the fifth solution through a 0.22 μm filter membrane or filter cartridge to obtain a sixth solution;
(7) filling the sixth solution into 10 ml-sized vials and applying half-stoppers; and
(8) transferring the aforementioned samples into the freeze-dryer, and lyophilizing according to the predetermined lyophilization curve: pre-freezing at −45° C. for 6 hours, followed by vacuum-pumping and heating to −15° C. for sublimation drying for 15 hours, then heating to 30° C. for desorption drying for 6 hours. Once lyophilization is finished, fully stoppering the vials, removing the vials from the freeze-dryer, crimping the caps, and obtaining lyophilized powder for inhalation containing Genipin-1-β-D-gentiobioside.

### Formulation Embodiment 6: Inhalation Administration—Lyophilized Powder for Inhalation

A lyophilized powder for inhalation containing Genipin-1-β-D-gentiobioside has the following formulation: 200 mg of Genipin-1-β-D-gentiobioside, 0.1% (w/v) citric acid, disodium hydrogen phosphated to adjust the pH to 7.0, and solvent to a total volume of 5 ml.

The preparation method of the aforementioned lyophilized powder for inhalation comprises the following steps:
(1) measuring 90% (v/v) of total solution volume required for preparation using water for injection to obtain a first solution;
(2) adding the citric acid and disodium hydrogen phosphated into the first solution at a temperature of 80° C., adjusting the pH to 7.0, and stirring uniformly to obtain a second solution;
(3) adding Genipin-1-β-D-gentiobioside into the second solution, and stirring uniformly to obtain a third solution;
(4) measuring the pH, and if the pH is not within the range of 6.9 to 7.1, adding an appropriate amount of disodium hydrogen phosphated again to adjust the solution's pH to 7.0 to obtain a fourth solution;
(5) adding solvent into the fourth solution to adjust the volume to the total required volume for the preparation, and stirring uniformly to obtain a fifth solution;
(6) filtering the fifth solution through a 0.22 μm filter membrane or filter cartridge to obtain a sixth solution;
(7) filling the sixth solution into 10 ml-sized vials and applying half-stoppers; and
(8) transferring the aforementioned samples into the freeze-dryer, and lyophilizing according to the predetermined lyophilization curve: pre-freezing at −45° C. for 6 hours, followed by vacuum-pumping and heating to −15° C. for sublimation drying for 25 hours, then heating to 20° C. for desorption drying for 10 hours. Once lyophilization is finished, fully stoppering the vials, removing the vials from the freeze-dryer, crimping the caps, and obtaining lyophilized powder for inhalation containing Genipin-1-β-D-gentiobioside.

For the samples obtained from the Formulation Embodiments 4-6, upon use, place the nebulizer (which can operate based on principles such as air compression, vibrating mesh, or ultrasonics) on a flat surface. During operation, keep the device away from textiles to avoid blocking the air inlet with textile fibers. After correctly installing the nebulization cup according to the user manual, open the medicine package, take out the vial, draw 1-5 ml of sterile water for injection using a syringe, inject the sterile water into the vial, and shake well until the lyophilized powder is completely dissolved. Then, draw the solution into the syringe and transfer it to the nebulization cup. Sit or stand upright to ensure normal breathing. After confirming that the nebulization mask covers your nose and mouth or the nebulization mouthpiece is placed in your mouth, press the nebulization button to start the nebulization process and continue inhaling until no more droplets are emitted. To reduce the risk of infection, illness, or contamination, clean and disinfect the nebulizer according to the instructions after treatment.

Stability testing experiments have shown that the lyophilized powders obtained from the three Formulation Embodiments are stable in terms of content and related substances, and their nebulization characteristics meet the requirements.

### Formulation Embodiment 7: Injection Administration—Lyophilized Powder

A lyophilized powder for injection containing Genipin-1-β-D-gentiobioside has the following formulation: 100 mg of Genipin-1-β-D-gentiobioside, sodium chloride to adjust the osmolarity to isotonicity, sulfuric acid/sodium hydroxide to adjust the pH to 6.0, and water for injection to a total volume of 3 ml.

The preparation method of lyophilized powder for injection comprises the following steps:
(1) measuring 40% (v/v) of total solution volume required for preparation using water for injection to obtain a first solution;
(2) adding the sodium chloride into the first solution at a temperature of 20° C., and stirring uniformly to obtain a second solution;
(3) adding the sodium hydroxide into the second solution, adjusting the pH to 6.0, and stirring uniformly to obtain a third solution;
(4) adding Genipin-1-β-D-gentiobioside into the third solution, and stirring uniformly to obtain a fourth solution;
(5) if the pH is not within the range of 5.9 to 6.1, adding an appropriate amount of sulfuric acid or sodium hydroxide again to adjust the solution's pH to 6.0 to obtain a fifth solution;
(6) adding water for injection into the fifth solution to adjust the volume to the total required volume for the preparation, and stirring uniformly to obtain a sixth solution;
(7) filtering the sixth solution through a 0.22 μm filter membrane or filter cartridge to obtain a seventh solution;
(8) filling the seventh solution into 10 ml-sized vials and applying half-stoppers; and
(9) transferring the aforementioned samples into the freeze-dryer, and lyophilizing according to the predetermined lyophilization curve: pre-freezing at −45° C. for 6 hours, followed by vacuum-pumping and heating to −20° C. for sublimation drying for 14 hours, then heating to −10° C. and continuing sublimation drying for 12 hours. After the sublimation drying is completed, heating to 20° C. for desorption drying and keeping for 4 hours. Once lyophilization is finished, fully sealing the vials, removing the vials from the freeze-dryer, and crimping the caps.

### Formulation Embodiment 8: Injection Administration—Lyophilized Powder

A lyophilized powder for injection containing Genipin-1-β-D-gentiobioside has the following formulation: 200 mg of Genipin-1-β-D-gentiobioside, sodium chloride to adjust the osmolarity to isotonicity, and water for injection to a total volume of 5 ml.

The preparation method of lyophilized powder for injection comprises the following steps:
(1) measuring 90% (v/v) of total solution volume required for preparation using water for injection to obtain a first solution;
(2) adding the sodium chloride into the first solution at a temperature of 50° C., and stirring uniformly to obtain a second solution;
(3) adding Genipin-1-β-D-gentiobioside into the second solution, and stirring uniformly to obtain a third solution;
(4) adding water for injection into the third solution to adjust the volume to the total required volume for the preparation, and stirring uniformly to obtain a fourth solution;
(5) filtering the fourth solution through a 0.22 μm filter membrane or filter cartridge to obtain a fifth solution;
(6) filling the fifth solution into 10 ml-sized vials and applying half-stoppers; and
(7) transferring the aforementioned samples into the freeze-dryer, and lyophilizing according to the predetermined lyophilization curve: pre-freezing at −45° C. for 6 hours, followed by vacuum-pumping and heating to −20° C. for sublimation drying for 20 hours, then heating to −10° C. and continuing sublimation drying for 16 hours. After the sublimation drying is completed, heating to 30° C. for desorption drying and keeping for 8 hours. Once lyophilization is finished, fully sealing the vials, removing the vials from the freeze-dryer, crimping the caps, and obtaining lyophilized powder for inhalation containing Genipin-1-β-D-gentiobioside.

### Formulation Embodiment 9: Injection Administration—Lyophilized Powder

A lyophilized powder for injection containing Genipin-1-β-D-gentiobioside has the following formulation: 30 mg of Genipin-1-β-D-gentiobioside, sodium dihydrogen phosphate and disodium hydrogen phosphate to adjust the pH to 7.0, sodium chloride to adjust the osmolarity to isotonicity, and water for injection to a total volume of 5 ml.

The preparation method of lyophilized powder for injection comprises the following steps:
(1) measuring 60% (v/v) of total solution volume required for preparation using water for injection to obtain a first solution;
(2) adding the sodium chloride into the first solution at a temperature of 80° C., and stirring uniformly to obtain a second solution;
(3) adding the sodium dihydrogen phosphate and disodium hydrogen phosphate into the second solution, adjusting the pH to 7.0, and stirring uniformly to obtain a third solution;
(4) adding Genipin-1-β-D-gentiobioside into the third solution, and stirring uniformly to obtain a fourth solution;
(5) measuring the pH, and if the pH is not within the range of 6.9 to 7.1, adding an appropriate amount of sodium dihydrogen phosphate/disodium hydrogen phosphate again to adjust the solution's pH to 7.0 to obtain a fifth solution;
(6) adding water for injection into the fifth solution to adjust the volume to the total required volume for the preparation, and stirring uniformly to obtain a sixth solution;
(7) filtering the sixth solution through a 0.22 μm filter membrane or filter cartridge to obtain a seventh solution;
(8) filling the seventh solution into 10 ml-sized vials and applying half-stoppers; and
(9) transferring the aforementioned samples into the freeze-dryer, and lyophilizing according to the predetermined lyophilization curve: pre-freezing at −45° C. for 6 hours, followed by vacuum-pumping and heating to −20° C. for sublimation drying for 18 hours, then heating to −10° C. and continuing sublimation drying for 15 hours. After the sublimation drying is completed, heating to 20˜30° C. for desorption drying and keeping for 7 hours. Once lyophilization is finished, fully sealing the vials, removing the vials from the freeze-dryer, crimping the caps, and obtaining lyophilized powder for inhalation containing Genipin-1-β-D-gentiobioside.

For the lyophilized powder obtained from the Formulation Embodiments 7-9, upon use, open the medicine package, take out the vial, draw 1-5 ml of sterile water for injection using a syringe, inject the sterile water into the vial, and shake well until the lyophilized powder is completely dissolved. Subsequently, it can be administered by intramuscular injection, intravenous injection, or mixed with other infusion solutions for intravenous drip administration.

Stability testing experiments have shown that the lyophilized powders obtained from the Formulation Embodiments 7-9 are stable in terms of content and related substances when stored at 25° C. for 6 months.

### Pharmacological Experiment Embodiment 1: Protective Effect of Intravenous Infusion of Genipin-1-β-D-Gentiobioside Against Death in a COVID-19 Pneumonia Mouse Model

### 1 Experimental Materials

1.1 Test Drug: Genipin-1-β-D-gentiobioside. Appearance: white powder; Solubility: extremely soluble in water. Dosage for mice: 37.5 mg/kg and 75 mg/kg, administered by intraperitoneal injection once daily for 5 consecutive days.

1.2 Experimental Animals: human angiotensin-converting enzyme 2 (hACE2) transgenic C57BL/6 mice, aged 6˜7 weeks, weighing 18˜25 g, a total of 32 mice provided by GemPharmatech Co., Ltd., with a license number of SCXK (Su) 2018-008.

1.3 Experimental Conditions: The experiment was conducted in the ABSL-3 laboratory of Guangzhou Institute of Respiratory Health.

### 2 Experimental Methods

2.1 Grouping and Drug Administration: hACE2 transgenic C57BL/6 mice were divided into four groups, namely the blank control group, the SARS-CoV-2 infection group, the Genipin-1-β-D-gentiobioside (37.5 mg/kg) treatment group, and the Genipin-1-β-D-gentiobioside (75 mg/kg) treatment group, with 8 mice in each group. Except for the normal group, which received intranasal administration of Phosphate Buffered Saline (PBS), the other groups were infected with 10⁴ Plaque Forming Unit (PFU) of Severe Acute Respiratory Syndrome Coronavirus 2 (SARS-CoV-2) virus via intranasal instillation. Two hours' post-infection, the treatment groups received intraperitoneal injections of the drug once daily for 5 consecutive days. Mouse deaths were recorded daily after infection, and the 5-day mortality rate was calculated. At the end of the experiment, the lung tissues were collected and homogenized for virus titer detection.

### 2.2 Virus Titer Detection in Mouse Lung Tissue Homogenates:

After excising the mouse lung tissues, they were placed in a Petri dish, cut into small pieces, transferred to a homogenization tube, and diluted with physiological saline at a ratio of 1:10 (w/v). The homogenization was performed at 8000 rpm/min for 10 min, with all operations conducted on ice. The homogenate was then transferred to a 1.5 mL EP tube and centrifuged at 10000 rpm for 10 min at 4° C. The supernatant was collected, aliquoted, and stored at −80° C. for future use.

The well-grown VERO E6 cells were inoculated into a 96-well plate at a density of 1×10⁴ cells per well and cultured for another 24 hours. After the cells adhered to the wall and formed a complete monolayer, the supernatant was discarded, and the cells were washed twice with PBS. The frozen lung homogenate supernatants were thawed and then serially diluted 10-fold to obtain five concentrations ranging from 10⁻¹ to 10⁻⁵. The diluted lung homogenate supernatants from each mouse group were added to the 96-well plate, with 100 μL of lung supernatant added to each well. Meanwhile, blank control wells were set up by adding cell culture medium. Each concentration was tested in quadruplicate. The plate was then placed in an incubator for cultivation. Over the next 4 days, the cells were observed daily for Cytopathic Effects (CPE). The number of wells showing CPE at each concentration gradient was recorded, and the Tissue Culture Infectious Dose 50% (TCID₅₀) value for VERO E6 cells was calculated.

### 3 Experimental Results

### 3.1 Protective Effect of Genipin-1-β-D-gentiobioside Against Death Caused by SARS-CoV-2 Infection-Induced Pneumonia in Mice

TABLE 1
Protective Effect of Genipin-1-β-D-gentiobioside
Against Death Caused by SARS-CoV-2 Infection in Mice

| | | Number | Number | Number | Death | Survival |
|---|---|---|---|---|---|---|
| | Dose | of | of | of | Rate | Rate |
| Group | (mg/kg) | Animals | Deaths | Survivors | (%) | (%) |
| Blank Control | — | 8 | 0 | 8 | 0 | 100 |
| Group | | | | | | |
| Model Control | — | 8 | 6 | 2 | 75 | 25 |
| Group | | | | | | |
| Genipin-1-β-D- | 37.5 | 8 | 3 | 5 | 37.5 | 62.5 |
| gentiobioside | 75 | 8 | 2 | 6 | 25 | 75 |

The results in Table 1 indicate that during the experiment, there were no deaths in the blank control group. After SARS-CoV-2 virus infection, the death rate in the model control group was 75%. When administered intraperitoneally once daily for five consecutive days at doses of 37.5 mg/kg and 75 mg/kg, Genipin-1-β-D-gentiobioside significantly reduced the number of deaths in mice infected with SARS-CoV-2, with death rates of 37.5% and 25%, respectively. These findings suggest that Genipin-1-β-D-gentiobioside has a significant protective effect against death caused by SARS-CoV-2 infection in mice and demonstrates a good dose-response relationship.

3.2 Effect on Virus Titer in the Lung Tissue of Mice Infected with SARS-CoV-2

As shown in the sole FIGURE, the results indicate that after virus infection, there was significant virus replication in the lung tissue of mice in the model control group. Administration of Genipin-1-β-D-gentiobioside at doses of 37.5 mg/kg and 75 mg/kg significantly reduced the virus titer in the lung tissue of mice compared to the model control group, with statistically significant differences (P<0.05, P<0.01, respectively). Furthermore, a good dose-response relationship was observed.

### Pharmacological Experiment Embodiment 2: Therapeutic Effect of Intravenous Administration of Genipin-1-β-D-gentiobioside on Mice Pneumonia Models Infected with Human Coronaviruses 229E and OC43

### 1 Experimental Materials

1.1 Test Drug: Genipin-1-β-D-gentiobioside. Appearance: white powder; Solubility: extremely soluble in water. Dosage for mice: 150 mg/kg, 75 mg/kg, 37.5 mg/kg, administered intravenously once daily for four consecutive days.

1.2 Positive Drug: Chloroquine Phosphate Tablets, manufactured by Sichuan Sunnyhope Pharmaceutical Co. Ltd., batch number: 2002114, manufacturing date: Feb. 26, 2020, expiration date: January 2022. Specification: 0.25 g/tablet. Dosage and administration: 0.5 g/60 kg/day, orally administered.

### 1.3 Experimental Animals

TABLE 2
Experimental Animals Table

| Species | Grade | Weight | Quantity | Gender | Certificate Number |
|---|---|---|---|---|---|
| BALB/c Mice | SPF | 13~15 | 120 | Half male | 110011211103579234/155 |
| | | | | and half | 110011211110038632/38717 |
| | | | | female | |

| | |
|---|---|
| Source | Beijing Vital River Laboratory Animal Technology Co., Ltd. |
| License Number | SCXK(Beijing)2019-0009 |

1.4 Virus Strains and Cells: Human Coronavirus 229E (HCoV-229E) provided by the Institute of Medical Biotechnology (IMB), Chinese Academy of Medical Sciences; Human Coronavirus HCoV-OC43 purchased from the American Type Culture Collection (ATCC). Propagated in our laboratory and stored at −80° C. for future use. Human embryonic lung fibroblast cells (MRC-5) purchased from BeNa Culture Collection. Propagated in our laboratory and stored in liquid nitrogen for future use.

### 1.5 Experimental Instruments

TABLE 3
Experimental Instruments Table

| | | | Instrument |
|---|---|---|---|
| Instrument Name | Model | Manufacturer | Purpose |
| Biosafety Cabinet | Thermo | Thermo Fisher Scientific Inc. | Animal virus |
| Type A2 | MSC1.8 | | infection |
| Electronic Balance | YP1002 | Shanghai Yueping Scientific Instrument | Weighing |
| | | Co., Ltd. | Mouse Body |
| | | | Weight |
| Electronic Balance | AL204 | Mettler-Toledo Instruments (Shanghai) | Weighing tissue |
| | | Co., Ltd. | weight |
| IVC Mouse Cage | ZJ-4 | Suzhou Fengshi Co., Ltd. | Housing mice |

1.6 Experimental Site: ABSL-2 Laboratory, Institute of Chinese Materia Medica, China Academy of Chinese Medical Sciences.

### 2 Experimental Methods

### 2.1 Drug Dosage Design and Preparation

Test Drug: The dosages for mice were set at 150 mg/kg/day, 75 mg/kg/day, and 37.5 mg/kg/day.

The drug was dissolved in physiological saline to prepare corresponding solutions. During the experiment, the solutions were administered to mice via intravenous injection at a rate of 2 ml/10 g/day, once daily for four consecutive days.

Chloroquine Phosphate Tablets: The clinical dosage for humans is 0.5 g/60 kg/day, orally administered.

For mice, the clinical dosage was converted to a mouse dosage using the following calculation: 0.5 g/60 kg/day×1=0.09 g/kg/day.

The drug was administered to mice via gavage at a volume of 20 ml/kg/day.

The concentration of the solution was prepared as follows: 0.09 g/kg/day÷20 ml/kg/day=0.0045 g/ml.

### 2.2 Virus Passage

Monolayer MRC-5 cells in a 25 cm² culture flask were taken, and the culture medium was discarded. After rinsing the cell surface three times with cell maintenance medium, 5 ml of cell maintenance medium was added, followed by the addition of 200 μl of HCoV-229E or OC43 virus solution. The flasks were then incubated in a 37° C. incubator with 5% CO₂ for 72 to 96 hours. The cell pathology was observed daily under an inverted microscope until 80% of the cells showed obvious cytopathic effects (CPE). Subsequently, the cell culture flasks were stored in a −80° C. freezer, and the virus solution was subjected to three freeze-thaw cycles before being used for virus titer determination.

### 2.3 Virus Titer Determination

Monolayer MRC-5 cells in a 96-well plate were taken, and the culture medium was discarded. After rinsing the cells three times with cell maintenance medium, different dilutions of HCoV-229E or OC43 virus solution were inoculated at 10-fold increments (101 to 108), resulting in eight dilutions, with 100 μl per well. Each dilution was tested in quadruplicate, and normal cell controls were set up. The 96-well plate was incubated in a 37° C. incubator with 5% CO₂ for 72 to 96 hours. The cell pathology was observed daily under an inverted microscope, and the CPE in each well was recorded. The 50% tissue culture infective dose (TCID₅₀) was calculated using the Reed-Muench method.

2.4 Establishment of Mouse Pneumonia Model Infected with Human Coronaviruses and Drug Administration

BALB/c mice were randomly divided into a normal control group, a model control group, a chloroquine phosphate control group, and three dose groups of Genipin-1-β-D-gentiobioside, with 10 mice in each group, half male and half female. Except for the normal control group, the mice in the other groups were lightly anesthetized with ether and infected intranasally with 100 TCID₅₀ of HCoV-229E or OC43, 50 μl per mouse, once every other day for a total of two infections. On the day of the initial infection, the drug administration groups began intravenous injection once daily for four consecutive days. On the fifth day, the mice were weighed, and their lungs were dissected, weighed, and used to calculate the lung index and inhibition rate.

$$Lung index = ( Lung wet weight ( g ) / Body weight ( g ) ) \times 100$$

$$Inhibition rate of lung index = ( Lung index of model control group - Lung index of drug administration group ) / ( Lung index of model control group - Lung index of normal control group ) \times 100 \%$$

### 3 Experimental Results

TABLE 4
Therapeutic Effect of Genipin-1-β-D-gentiobioside
Administered by Intravenous Injection on a Mouse
Pneumonia Model Infected with Human Coronavirus 229E

| | | Number | Lung Index | |
|---|---|---|---|---|
| | | of | (g lung weight × | Inhibition |
| | Dosage | Animals | 100 g/body | Rate |
| Group | (mg/kg/d) | (n) | weight) | (%) |
| Normal | — | 10 | 0.89 ± 0.05 | — |
| Control Group | | | | |
| Model Control | — | 10 | 1.14 ± 0.16^{##} | |
| Group | | | | |
| Chloroquine | 90 | 10 | 0.90 ± 0.08** | 93.99 |
| Control Group | | | | |
| Genipin-1-β- | 150 | 10 | 0.89 ± 0.06** | 99.06 |
| D- | 75 | 10 | 0.96 ± 0.12** | 71.04 |
| gentiobioside | 37.5 | 10 | 0.95 ± 0.11** | 75.12 |

Note:
Compared with the normal control group,
^{##}P < 0.01,
^{#}P < 0.05; Compared with the model control group,
**P < 0.01,
*P < 0.05.

The results in Table 4 indicate that after mice were infected with human coronavirus 229E, their lung index significantly increased, with a significant difference compared to the normal control group (P<0.01). When Genipin-1-β-D-gentiobioside was administered intravenously for 4 days starting on the day of infection, all three tested doses significantly reduced the lung index in mice infected with 229E, with significant differences compared to the model control group (P<0.01). The inhibition rates of the lung index were 99.06%, 71.04%, and 75.12%, respectively. The drug efficacy was comparable to that of chloroquine phosphate.

TABLE 5
Therapeutic Effect of Genipin-1-β-D-gentiobioside
Administered by Intravenous Injection on a Mouse
Pneumonia Model Infected with Human Coronavirus OC43

| | | Number | | |
|---|---|---|---|---|
| | | of | Lung Index | Inhibition |
| | Dosage | Animals | (g lung weight × 100 | Rate |
| Group | (mg/kg/d) | (n) | g/body weight) | (%) |
| Normal Control | — | 10 | 0.69 ± 0.06 | — |
| Group | | | | |
| Model Control | — | 10 | 0.87 ± 0.09^{##} | |
| Group | | | | |
| Chloroquine | 90 | 10 | 0.80 ± 0.05* | 42.72 |
| Control Group | | | | |
| Genipin-1-β-D- | 150 | 10 | 0.80 ± 0.07* | 39.93 |
| gentiobioside | 75 | 10 | 0.80 ± 0.05** | 42.34 |
| | 37.5 | 10 | 0.80 ± 0.08* | 41.59 |

Note:
Compared with the normal control group,
^{##}P < 0.01,
^{#}P < 0.05; Compared with the model control group,
**P < 0.01,
*P < 0.05.

The results in Table 5 indicate that after mice were infected with human coronavirus OC43, their lung index significantly increased, with a significant difference compared to the normal control group (P<0.01). When Genipin-1-β-D-gentiobioside was administered intravenously for 4 days starting on the day of infection, all three tested doses significantly reduced the lung index in mice infected with OC43, with significant differences compared to the model control group (P<0.05, P<0.01). The inhibition rates of the lung index were 39.93%, 42.34%, and 41.59%, respectively. The drug efficacy was comparable to that of chloroquine phosphate.

### Pharmacological Experiment Embodiment 3: Therapeutic Effect of Genipin-1-β-D-gentiobioside Administered by Nebulization on Mouse Pneumonia Models Infected with Human Coronaviruses 229E and OC43

### 1 Experimental Materials

1.1 Test Drug: Genipin-1-β-D-gentiobioside, Batch Number: 20210106; Appearance: white powder; Solubility: extremely soluble in water.

1.2 Experimental Animals: BALB/c Mice, SPF Grade, Weighing 13˜15 g, 140 Animals in Total, with an Equal Number of Males and Females. Sourced from Beijing Vital River Laboratory Animal Technology Co., Ltd.

1.3 Virus Strains and Cells: Human Coronavirus 229E (HCoV-229E) and Human Coronavirus GC43 (HCoV-OC43), purchased from the American Type Culture Collection (ATCC), with a TCID₅₀ of 10⁻⁴. Propagated in our laboratory and stored at −80° C. for future use. Human embryonic lung fibroblast cells (MRC-5) purchased from BeNa Culture Collection. Propagated in our laboratory and stored in liquid nitrogen for future use.

### 1.4 Experimental Reagents:

TABLE 6
Experimental Reagents Table

| | | Batch | |
|---|---|---|---|
| Reagent Name | Manufacturer | Number | Reagent Purpose |
| Isoflurane | Shanghai Yuyan | S200106 | Mouse Anesthesia |
| | Instruments Co., Ltd. | | |
| Distilled Water | Guangzhou Watson's | GB19298 | Nebulization |
| | Food & Beverage Co., | | Administration to |
| | Ltd. | | Mice |
| Trizol Reagent | ambion by life | 279505 | Viral Nucleic Acid |
| | technologies | | Detection |
| (HCoV-229E)Real Time | Shanghai Zhijiang | P20210101 | Viral Nucleic Acid |
| RT-PCR kit | Biotechnology Co., Ltd. | | Detection |
| Eight-Strip | life technologies | I42C0Q523 | Viral Nucleic Acid |
| | | | Detection |
| Mouse IL-6 ELISA Kit | Shanghai Enzyme-linked | 05/2022 | Detection of Lung |
| | Biotechnology Co., Ltd. | | Tissue Factors in |
| | | | Mice |
| Mouse IL-10 ELISA Kit | Shanghai Enzyme-linked | 05/2022 | Detection of Lung |
| | Biotechnology Co., Ltd. | | Tissue Factors in |
| | | | Mice |
| Mouse TNF-α ELISA | Shanghai Enzyme-linked | 05/2022 | Detection of Lung |
| Kit | Biotechnology Co., Ltd. | | Tissue Factors in |
| | | | Mice |

### 1.5 Experimental Instruments:

TABLE 7
Experimental Instruments Table

| | | | Instrument |
|---|---|---|---|
| Instrument Name | Manufacturer | Model | Purpose |
| Biosafety Cabinet | Biobase Biodustry (Shandong) | 11229BBC86 | Mouse Infection |
| Type A2 | Co., Ltd. | | Procedure |
| Biosafety Cabinet | Biobase Biodustry (Shandong) | 11229BBC86 | Protection |
| Type A2 | Co., Ltd. | | During Mouse |
| | | | Nebulization |
| Biosafety Cabinet | Thermo Fisher Scientific Inc. | 1379 | Mouse |
| Type A2 | | | Dissection |
| IVC Mouse Cage | Shenzhen Hongtengkeji BIO- | HT-1202A | Mouse Housing |
| | TECH Co., Ltd. | | |
| Animal Nasal | Melton Lab Co., Ltd. | Inhalogic | Nebulization |
| Inhalation Exposure | | NIES | Administration |
| System | | | to Mice |
| Nebulizer | PARI, Germany | Blue Core | Nebulization |
| | | | Administration |
| | | | to Mice |
| Multi-Mode | BioTek, USA | Synergy H1 | Detection with |
| Microplate Reader | | | ELISA Kits |
| Microplate Incubator | Hangzhou Allsheng | Hangzhou | Detection with |
| | Instruments Co., Ltd. | Allsheng | ELISA Kits |
| | | MK100-4A | |
| Real-Time PCR | Applied Biosystems, USA | QuantStudio5 | Nucleic Acid |
| Instrument | | | Detection |
| Eight-Strip Tube Mini | Kylin-Bell Lab Instruments | LX-300 | Nucleic Acid |
| Centrifug | | | Detection |
| Mini Vortex Mixer | Haimen Kylin-Bell Lab | OL-901 | Nucleic Acid |
| | Instruments Co., Ltd. | | Detection |
| High-Throughput | Ningbo Scientz Biotechnology | Scientz-48L | Lung Tissue |
| Tissue Grinder | Co., Ltd. | | Grinding |
| Electronic Balance | Sartorius | BSA3202S- | Weighing mouse |
| | | CW | weight |
| Electronic Balance | Sartorius | BSA323S- | Weighing |
| | | CW | Mouse Lung |
| | | | Weight |

### 1.6 Experimental Site: ABSL-2 Laboratory, Institute of Chinese Materia Medica, China Academy of Chinese Medical Sciences.

### 2 Experimental Methods

### 2.1 Drug Dosage Design and Preparation

### Test Drugs:

1. Genipin-1-β-D gentiobioside 75 mg/ml group, median particle size is 2.02±0.06 μm; Genipin-1-β-D gentiobioside 37.5 mg/ml group, median particle size is 2.12±0.08 μm; Genipin-1-β-D gentiobioside 18.755 mg/ml group, median particle size is 2.01±0.09 μm; and Genipin-1-β-D gentiobioside 9.375 mg/ml group, median particle size is 2.07±0.06 μm. The nebulization time for each group is 25 minutes.

2. Chloroquine Phosphate Tablets, with a clinical dosage of 0.5 g/60 kg/day, administered orally. For mice, the dosage is calculated as 0.5 g/60 kg/day×11=0.09 g/kg/day. The administration volume for mice is 20 ml/kg/day, given by gavage. The preparation concentration is calculated as 0.09 g/kg/day÷20 ml/kg/day=0.0045 g/ml.

### 2.2 Grouping and Drug Administration

Seventy Institute of Cancer Research Mouse (ICR) mice were randomly divided into seven groups based on body weight: a normal control group, a model control group, a chloroquine phosphate control group, and four Genipin-1-β-D-gentiobioside dosage groups (75 mg/ml, 37.5 mg/ml, 18.75 mg/ml, and 9.375 mg/ml), with ten mice in each group. Except for the normal control group, the other mice were lightly anesthetized with isoflurane and infected intranasally with 50 μl of 100 TCID₅₀ coronavirus strain 229E or OC43. The infection was repeated every other day. Nebulization administration began on the same day as the first infection, with a flow rate of 7.5 L/min. The four Genipin-1-β-D-gentiobioside dosage groups were nebulized for 25 minutes each. The chloroquine phosphate control group was administered 0.2 ml/10 g body weight by gavage once daily for four consecutive days. The normal control group and the model control group were nebulized with distilled water for 25 minutes under the same conditions. On the fifth day, the mice were dissected, and the following indicators were measured:

\textcircled{1} After weighing, the mice were dissected, and their whole lungs were extracted and weighed to calculate the lung index and inhibition rate.

\textcircled{2} The left lung lobes were used for viral nucleic acid detection, while the right lung lobes were used for the quantification of inflammatory factors: IL-6, IL-10, and TNF-α.

The results were statistically analyzed using the t-test for comparisons between groups.

$$Lung index = ( Lung wet weight ( g ) / Body weight ( g ) ) \times 100$$

$$Inhibition rate of lung index = ( Lung index of model control group - Lung index of drug administration group ) / ( Lung index of model control group - Lung index of normal control group ) \times 100 \%$$

### 2.3 Detection of Viral Load in Lung Tissue (RT-PCR Method)

### \textcircled{1} Nucleic Acid Lysis Treatment

After dissecting the mice, the lung tissues were collected and stored in separate containers at −80° C. in a low-temperature freezer. The lung tissues were then removed from the −80° C. freezer and placed in a clean mortar. A small amount of liquid nitrogen was poured into the mortar, and the tissues were ground into a powder using a pestle. The powder was collected in a 1.5 ml centrifuge tube, and 1 ml of TRIzol Reagent was immediately added. The tube was gently tapped at the bottom to mix the sample and ensure it was resuspended as soon as possible. The centrifuge tube was then placed horizontally at room temperature and incubated for 20 minutes. Next, it was centrifuged at 4° C. and 12,000 rpm for 10 minutes. The clear supernatant was transferred to a new 1.5 ml centrifuge tube, and 0.2 ml of chloroform was added. The tube was tightly capped, vigorously shaken for 15 seconds, and then incubated at room temperature for 2-3 minutes until the liquid separated into layers. After centrifuging at 4° C. and 12,000 rpm for 15 minutes, the transparent supernatant was carefully transferred to a new 1.5 ml centrifuge tube. Then, 0.5 ml of isopropanol was added, mixed well, and incubated at room temperature for 30 minutes. Following centrifugation at 4° C. and 12,000 rpm for 10 minutes, the supernatant was discarded. The precipitate was gently washed with 1 ml of 75% ethanol (to allow the white precipitate to float slightly), and then centrifuged at 4° C. and 7,500 rpm for 5 minutes. The supernatant was completely aspirated, and the RNA precipitate was briefly dried for 5-10 minutes. The precipitate was then dissolved in 20 μl of DEPC (Diethylpyrocarbonate)-treated water and stored in a −80° C. low-temperature freezer.

### \textcircled{2} Nucleic Acid Quantification

Treatment of Control Nucleic Acid: DEPC-H₂O was used as a negative control. For the positive control, a series of four concentrations were prepared by gradient dilution at 10⁷, 10⁶, 10⁵, and 10⁴ copies/ml.

Reagent Preparation: Mix n×18 μl of HCoV-229E nucleic acid fluorescent PCR detection mixture, n×1 μl of internal control, and n×1 μl of RT-PCR enzyme (where n is the number of reaction tubes). Shake vigorously for a few seconds and centrifuge at 3000 rpm for a few seconds.

Sample Addition: Dispense 20 μl of the above mixture into each PCR tube. Then, add 5 μl of sample nucleic acid extract, DEPC-H₂O, and positive control to the respective PCR tubes. Tightly close the tube caps, centrifuge for a few seconds to ensure all liquids are at the bottom, and immediately proceed with PCR amplification.

PCR Amplification: Place the reaction tubes in a quantitative fluorescent PCR instrument. Set the cycling parameters as follows: 45° C. for 10 minutes; 95° C. for 15 minutes; then 40 cycles of 95° C. for 15 seconds→60° C. for 60 seconds. Single-point fluorescent detection is performed at 60° C., with a reaction volume of 25 μl.

Fluorescent Channel Selection: Select the FAM and HEX/VIC/JOE channels for detection.

\textcircled{3} Calculation Method: A standard curve is plotted based on the Ct values of the positive controls at different concentrations. The viral nucleic acid concentration in the samples is then calculated using their respective Ct values.

### 3 Experimental Results

TABLE 8
Therapeutic Effect of Genipin-1-β-D-Gentiobioside Inhalation
Administration on HCOV-229E Infected Mouse Pneumonia Model

| | | | Number of | Lung Index | |
|---|---|---|---|---|---|
| | Inhalation | Dosage | Animals | (g lung weight × | Inhibition |
| Group | Time (min) | (mg/ml) | (n) | 100 g/body weight) | Rate (%) |
| Normal | 25 | — | 10 | 0.72 ± 0.08 | — |
| Control Group | | | | | |
| Model | 25 | — | 10 | 0.87 ± 0.05^{##} | — |
| Control Group | | | | | |
| Chloroquine | Intragastric | 90 mg/kg | 10 | 0.79 ± 0.05** | 56.72 |
| Control Group | administration | | | | |
| Genipin-1-β- | 25 | 75 | 10 | 0.71 ± 0.05** | 109.19 |
| D-gentiobioside | 25 | 37.5 | 10 | 0.77 ± 0.05** | 64.22 |
| | 25 | 18.75 | 10 | 0.77 ± 0.08** | 65.59 |
| | 25 | 9.375 | 10 | 0.77 ± 0.08** | 65.98 |

Note:
Compared with the normal control group,
^{##}P < 0.01,
^{#}P < 0.05; compared with the model control group,
**P < 0.01,
*P < 0.05.

The results in Table 8 indicate that after mice were infected with the human coronavirus 229E strain, the lung index of the model control group was significantly increased, with a significant difference compared to the normal control group (P<0.01). Starting on the day of infection, after 4 days of inhalation therapy with Genipin-1-β-D-Gentiobioside, all four dose groups of Genipin-1-β-D-Gentiobioside at 75 mg/ml, 37.5 mg/ml, 18.75 mg/ml, and 9.375 mg/ml significantly reduced the lung index, with significant differences compared to the model control group (P<0.01). The inhibition rates of the lung index were 109.19%, 64.22%, 65.59%, and 65.98%, respectively. The drug efficacy was stronger than that of chloroquine phosphate.

TABLE 9
Effect of Genipin-1-β-D-Gentiobioside Inhalation Administration
on Viral Load in Lung Tissue of HCOV-229E Infected Mice

| | | | Number of | |
|---|---|---|---|---|
| | Inhalation | Dosage | Samples | Viral Load |
| Group | Time (min) | (mg/ml) | (n) | (copies/ml) |
| Normal | 25 | — | 10 | 0.00 ± 0.00 |
| Control | | | | |
| Group | | | | |
| Model | 25 | — | 10 | 1.52 ± 0.14 × 10\hat{ }5^{##} |
| Control | | | | |
| Group | | | | |
| Chloroquine | Administration | 0.09 mg/kg | 10 | 6.60 ± 0.65 × 10\hat{ }4** |
| Control | by gavage | | | |
| Group | | | | |
| Genipin-1-β- | 25 | 75 | 10 | 7.17 ± 0.73 × 10\hat{ }4** |
| D-gentiobioside | 25 | 37.5 | 10 | 7.86 ± 0.87 × 10\hat{ }4** |
| | 25 | 18.75 | 10 | 7.54 ± 1.05 × 10\hat{ }4** |
| | 25 | 9.375 | 10 | 7.54 ± 1.11 × 10\hat{ }4** |

Note:
Compared with the normal group,
^{##}p < 0.01; Compared with the model group,
**p < 0.01.

The results in Table 9 indicate that there is no viral nucleic acid expression in the lung tissue of normal mice. After mice were infected with the human coronavirus 229E strain, the coronavirus nucleic acid expression in the lung tissue of mice was significant. Starting on the day of infection, after 4 days of inhalation therapy with Genipin-1-β-D-Gentiobioside, the coronavirus 229E nucleic acid expression levels in the lung tissue of mice in all four dose groups of Genipin-1-β-D-Gentiobioside at 75 mg/ml, 37.5 mg/ml, 18.75 mg/ml, and 9.375 mg/ml were significantly reduced, with significant differences compared to the model control group (P<0.01).

TABLE 10
Therapeutic Effect of Genipin-1-β-D-Gentiobioside Inhalation
Administration on HCOV-OC43 Infected Mouse Pneumonia Model

| | | | Number of | Lung Index | |
|---|---|---|---|---|---|
| | Inhalation | Dosage | Animals | (g lung weight × | Inhibition |
| Group | Time (min) | (mg/ml) | (n) | 100 g/body weight) | Rate (%) |
| Normal | 25 | — | 10 | 0.72 ± 0.07 | — |
| Control Group | | | | | |
| Model Control | 25 | — | 10 | 0.86 ± 0.06^{##} | — |
| Group | | | | | |
| Chloroquine | Administration | 90 mg/kg | 10 | 0.73 ± 0.11** | 96.56 |
| Control Group | by gavage | | | | |
| Genipin-1-β- | 25 | 75 | 10 | 0.72 ± 0.05** | 102.88 |
| D-gentiobioside | 25 | 37.5 | 10 | 0.74 ± 0.08** | 90.30 |
| | 25 | 18.75 | 10 | 0.76 ± 0.04** | 75.73 |
| | 25 | 9.375 | 10 | 0.79 ± 0.09* | 49.42 |

Note:
Compared with the normal control group,
^{##}P < 0.01,
^{#}P < 0.05; Compared with the model control group,
**P < 0.01,
*P < 0.05.

The results in Table 10 indicate that after mice were infected with the human coronavirus OC43 strain, the lung index of the model control group was significantly increased, with a significant difference compared to the normal control group (P<0.01). Starting on the day of infection, after 4 days of inhalation therapy with Genipin-1-β-D-Gentiobioside, all four dose groups of Genipin-1-β-D-Gentiobioside at 75 mg/ml, 37.5 mg/ml, 18.75 mg/ml, and 9.375 mg/ml significantly reduced the lung index of infected mice, with significant differences compared to the model control group (P<0.05, P<0.01). The inhibition rates of the lung index were 102.88%, 90.30%, 75.73%, and 49.42%, respectively.

TABLE 11
Effect of Genipin-1-β-D-Gentiobioside Inhalation Administration
on Viral Load in Lung Tissue of HCOV-OC43 Infected Mice

| | | | Number of | |
|---|---|---|---|---|
| | Inhalation | Dosage | Samples | Viral Load |
| Group | Time (min) | (mg/ml) | (n) | (copies/ml) |
| Normal | 25 | — | 10 | 0.00 ± 0.00 |
| Control | | | | |
| Group | | | | |
| Model | 25 | — | 10 | 6.18 ± 0.87 × 10\hat{ }3^{##} |
| Control | | | | |
| Group | | | | |
| Chloroquine | Intragastric | 90 mg/kg | 10 | 1.52 ± 0.46 × 10\hat{ }3** |
| Control | administration | | | |
| Group | | | | |
| Genipin-1-β- | 25 | 75 | 10 | 1.82 ± 0.37 × 10\hat{ }3** |
| D-gentiobioside | 25 | 37.5 | 10 | 2.65 ± 0.35 × 10\hat{ }3** |
| | 25 | 18.75 | 10 | 2.67 ± 0.44 × 10\hat{ }3** |
| | 25 | 9.375 | 10 | 5.76 ± 1.02 × 10\hat{ }3 |

Note:
Compared with the normal group,
^{##}p < 0.01; Compared with the model group,
**p < 0.01.

The results in Table 11 indicate that there is no viral nucleic acid expression in the lung tissue of normal mice. After mice were infected with the human coronavirus OC43 strain, the coronavirus nucleic acid expression in the lung tissue of mice in the model control group was significant. Starting on the day of infection, after 4 days of inhalation therapy with Genipin-1-O-D-Gentiobioside, the coronavirus nucleic acid expression levels in the lung tissue of mice in the three dose groups of Genipin-1-β-D-Gentiobioside at 75 mg/ml, 37.5 mg/ml, and 18.75 mg/ml were significantly reduced, with significant differences compared to the model control group (P<0.01).

### Pharmacological Experiment Embodiment 4: Effect of Genipin-1-β-D-Gentiobioside on Inflammatory Cytokines in Lung Tissue of Mice Infected with HCOV-229E

The inhalation administration samples mentioned above were used. The weight of the lung tissue was measured, and an appropriate amount of physiological saline was added to make a 10% tissue homogenate. After homogenizing the tissue using a high-throughput tissue grinder, it was centrifuged at 4° C. and 3000 rpm for 10 minutes using a low-temperature high-speed centrifuge. The supernatant was then aspirated, aliquotted, and stored at −80° C. for future use, avoiding repeated freeze-thaw cycles. For detection, the absorbance at 450 nm was measured using a microplate reader according to the kit instructions.

TABLE 12
Effect of Genipin-1-β-D-Gentiobioside on Cytokines in
Lung Tissue of Mice Infected with Human Coronavirus 229E

| | | Number of | | | |
|---|---|---|---|---|---|
| | Dosage | Animals | Cytokine Levels in Lung Tissue (pg/mL) | | |
| Group | (mg/ml) | (n) | TNF-α | IL-6 | IL-10 |
| Normal | — | 10 | 451.88 ± 37.88 | 101.12 ± 2.74 | 324.01 ± 30.35 |
| Control | | | | | |
| Group | | | | | |
| Model | — | 10 | 882.84 ± 51.88^{##} | 154.11 ± 13.28^{##} | 709.43 ± 76.51^{##} |
| Control | | | | | |
| Group | | | | | |
| Genipin-1-β- | 75 | 7 | 511.81 ± 51.82** | 98.31 ± 2.78** | 341.12 ± 29.07** |
| D gentiobioside | 37.5 | 9 | 513.28 ± 58.74** | 98.23 ± 3.42** | 334.12 ± 25.59** |
| | 18.75 | 10 | 503.00 ± 56.50** | 99.51 ± 5.24** | 330.61 ± 25.07** |
| | 9.375 | 8 | 511.16 ± 80.14** | 98.70 ± 4.56** | 331.34 ± 28.00** |

Compared with the normal control group,
^{##}P < 0.01,
^{#}P < 0.05; Compared with the model group,
**P < 0.01.

The results in Table 12 indicate that after modeling with human coronavirus 229E infection, the levels of TNF-α, IL-6, and IL-10 in the lung tissue of mice in the model group were significantly increased, with significant differences compared to the normal control group (P<0.01). Starting on the day of infection, after 4 days of inhalation administration of Genipin-1-β-D-Gentiobioside, all four dose groups were able to reduce the levels of TNF-α, IL-6, and IL-10 in the lung tissue of mice, with significant differences compared to the model group (P<0.01).

### Pharmacological Embodiment 5: Therapeutic Effect of Genipin-1-β-D-Gentiobioside Inhalation Administration on a Mouse Pneumonia Model Infected with Respiratory Syncytial Virus (RSV)

### 1 Experimental Materials

### 1.1 Test Drug

Genipin-1-β-D-Gentiobioside preparation; Specification: 5 ml; 375 mg; Batch Number: 230608; Appearance: light yellow liquid; Storage: room temperature.

### 1.2 Experimental Animals

Balb/c Mice, SPF Grade, Weight: 9-11 g, Quantity: 50 (25 males and 25 females), License Number: SCXK(Beijing)2016-0006.

### 1.3 Virus Strain

Human Respiratory Syncytial Virus (RSV), VR1580™, purchased from ATCC, passaged in our laboratory, stored at −80° C. for future use.

### 1.4 Experimental Instruments

TABLE 13
Experimental Instruments Table

| Instrument | | | |
|---|---|---|---|
| Name | Model | Manufacturer | Purpose |
| Biosafety | MSC1.2 | Thermo Fisher | Virus |
| Cabinet | | Scientific Inc. | Solution |
| Type A2 | | | Preparation |
| Biosafety | MSC1.8 | Thermo Fisher | Animal |
| Cabinet | | Scientific Inc. | Infection |
| Type A2 | | | |
| Electronic | BSA3202S-CW | Sartorius | Weighing |
| Balance | | Scientific | Weight |
| | | Instruments Co., | |
| | | Ltd. | |
| Electronic | Ltem-AR 1140 | Ohaus Corp. | Weighing |
| Analytical | | Brock. WJ. | Reagents |
| Balance | | USA | |
| Electronic | AL204 | Mettler-Toledo | Weighing |
| Balance | | Instruments Co., | Lung |
| | | Ltd. | Weight |
| Buxco Small | Buxco | DSI, USA | Drug |
| Animal | | | Administration |
| Nose-Only | | | in Mice |
| Exposure Tower | | | |

### 2 Experimental Methods and Results

### 2.1 Effect on Lung Index and Lung Index Inhibition Rate in Mice

Sixty BALB/c mice, weighing 10±1 g, were randomly divided into four groups: a normal control group, a model control group, a high-dose Genipin-1-β-D-gentiobioside group (75 mg/ml), and a low-dose Genipin-1-β-D-gentiobioside group (37.5 mg/ml). Each group consisted of 10 mice, with an equal number of males and females. Except for the normal control group, all mice were lightly anesthetized with isoflurane and infected with respiratory syncytial virus (RSV) via intranasal administration, with each mouse receiving 35 μl of the virus. On the day of infection, the treatment groups began inhalation administration. Each group received nebulization for 25 minutes once daily for four consecutive days. The normal control group and the model control group received nebulized saline under the same conditions. On the fifth day, after weighing, the mice were sacrificed by cervical dislocation, and their lung tissues were dissected, weighed, and used to calculate the lung index and lung index inhibition rate. The lung tissues were also retained for virus load, pathological examination, and inflammatory factor detection. The results were statistically analyzed using the t-test for intergroup comparisons.

$$Lung index = ( Lung wet weight ( g ) / Body weight ( g ) ) \times 100$$

$$Inhibition rate of lung index = ( Lung index of model control group - Lung index of drug administration group ) / ( Lung index of model control group - Lung index of normal control group ) \times 100 \%$$

TABLE 14
Therapeutic Effect of Genipin-1-β-D-Gentiobioside
Inhalation on a Mouse Pneumonia Model Infected with RSV

| | | | Number of | Lung Index | |
|---|---|---|---|---|---|
| | Dosage | Nebulization | Animals | (Lung weight/ | Inhibition |
| Group | (mg/ml) | Time (min) | (n) | 100 g body weight) | Rate (%) |
| Normal Control | — | — | 10 | 0.65 ± 0.07 | — |
| Group | | | | | |
| Model Control | — | — | 9 | 0.83 ± 0.08^{##} | — |
| Group | | | | | |
| Genipin-1-β- | 75 | 25 | 9 | 0.67 ± 014** | 88.35 |
| D-gentiobioside | 37.5 | 25 | 10 | 0.70 ± 0.09** | 68.79 |

Note:
Compared with the normal control group,
^{##}p < 0.01; Compared with the model control group,
**p < 0.01.

The results in Table 14 indicate that the lung index of mice in the RSV model control group was significantly increased, with a significant difference compared to the normal control group (p<0.01). After administering Genipin-1-β-D-gentiobioside at doses of 75 mg/ml and 37.5 mg/ml via nebulization for 25 minutes each time, once daily for four consecutive days, the lung index of the mice was significantly reduced, with a significant difference compared to the model control group (p<0.01). The lung index inhibition rates were 88.35% and 68.79%, respectively.

### 2.2 Effect on Virus Load in Mouse Lung Tissue

### Nucleic Acid Detection in Lung Tissue (RT-PCR Method)

### \textcircled{1} Nucleic Acid Lysis Treatment

After dissecting the mice, the lung tissues were collected and stored in separate containers at −80° C. in a low-temperature freezer. The lung tissues were then removed from the −80° C. freezer and placed in a clean mortar. A small amount of liquid nitrogen was poured into the mortar, and the tissues were ground into powder using a pestle. The powder was collected in a 1.5 ml centrifuge tube, and 1 ml of TRIzol Reagent was immediately added. The tube was gently tapped at the bottom to mix the sample and ensure it was resuspended as soon as possible. The centrifuge tube was then placed horizontally at room temperature and incubated for 20 minutes. Next, it was centrifuged at 4° C. and 12,000 rpm for 10 minutes. The clear supernatant was transferred to a new 1.5 ml centrifuge tube, and 0.2 ml of chloroform was added. The tube was tightly capped, vigorously shaken for 15 seconds, and then incubated at room temperature for 2-3 minutes until the liquid separated into layers. After centrifuging at 4° C. and 12,000 rpm for 15 minutes, the transparent supernatant was carefully transferred to a new 1.5 ml centrifuge tube. Then, 0.5 ml of isopropanol was added, mixed well, and incubated at room temperature for 30 minutes. Following centrifugation at 4° C. and 12,000 rpm for 10 minutes, the supernatant was discarded. The precipitate was gently washed with 1 ml of 75% ethanol (to allow the white precipitate to float slightly), and then centrifuged at 4° C. and 7,500 rpm for 5 minutes. The supernatant was completely aspirated, and the RNA precipitate was briefly dried for 5-10 minutes. The precipitate was then dissolved in 20 μl of DEPC (Diethylpyrocarbonate)-treated water and stored in a −80° C. low-temperature freezer.

### \textcircled{2} Nucleic Acid Quantification

Treatment of Control Nucleic Acid: DEPC-H₂O was used as a negative control. For the positive control, a series of four concentrations were prepared by gradient dilution at 10⁷, 10⁶, 10⁵, and 10⁴ copies/ml.

Reagent Preparation: Mix n×18 μl of RSV nucleic acid fluorescent PCR detection mixture, n×1 μl of internal control, and n×1 μl of RT-PCR enzyme (where n is the number of reaction tubes). Shake vigorously for a few seconds and centrifuge at 3000 rpm for a few seconds.

Sample Addition: Dispense 20 μl of the above mixture into each PCR tube. Then, add 5 μl of sample nucleic acid extract, DEPC-H₂O, and positive control to the respective PCR tubes. Tightly close the tube caps, centrifuge for a few seconds to ensure all liquids are at the bottom, and immediately proceed with PCR amplification.

PCR Amplification: Place the reaction tubes in a quantitative fluorescent PCR instrument. Set the cycling parameters as follows: 45° C. for 10 minutes; 95° C. for 15 minutes; then 40 cycles of 95° C. for 15 seconds→60° C. for 60 seconds. Single-point fluorescent detection is performed at 60° C., with a reaction volume of 25 μl.

Fluorescent Channel Selection: Select the FAM and HEX/VIC/JOE channels for detection.

Note: For ABI series PCR instruments, please ensure that both the passive reference and quencher are set to “none”.

Calculation Method: A standard curve is plotted based on the Ct values of the positive controls at different concentrations. The viral nucleic acid concentration in the samples is then calculated using their respective Ct values.

TABLE 15
Therapeutic Effect of Genipin-1-β-D-Gentiobioside
Inhalation on a Mouse Pneumonia Model Infected with RSV

| | | | Number of | | |
|---|---|---|---|---|---|
| | Dosage | Nebulization | Animals | Viral Load | Inhibition |
| Group | (mg/ml) | Time (min) | (n) | (copies/ng) | Rate |
| Normal Control | — | — | 5 | 4208.64 ± 3643.25 | |
| Group | | | | | |
| Model Control | — | — | 5 | 46413.93 ± 89545.46 | |
| Group | | | | | |
| Ribavirin Control | 82.5 | 25 | 5 | 17063.48 ± 16203.78 | 69.54 |
| Group | | | | | |
| Genipin-1-β- | 75 | 25 | 5 | 22745.57 ± 13287.06 | 56.08 |
| D-gentiobioside | 37.5 | 25 | 5 | 22127.79 ± 30384.54 | 57.43 |

The results in Table 15 indicate that the RSV virus load in the lung tissue of mice in the RSV model control group was significantly increased. After administering Genipin-1-β-D-gentiobioside at doses of 75 mg/ml and 37.5 mg/ml via nebulization for 25 minutes each time, once daily for four consecutive days, the RSV virus load in the lung tissue of mice was significantly reduced, and inhibition rates were 56.08% and 57.54%, respectively.

### Pharmacological Embodiment 6: Therapeutic Effect of Genipin-1-β-D-Gentiobioside Inhalation Administration on Mice Infected with Mycoplasma Pneumoniae

1.1 Test Drug: Genipin-1-β-D-gentiobioside preparation, specification: 5 ml; 375 mg, batch number: 230608.

1.2 Positive Drug: Azithromycin Capsules, batch number: 23032004, manufacturing date: Mar. 14, 2023, expiration date: February 2025. Manufactured by Sunflower Pharmaceutical Group Co., Ltd. Ingredients: The main ingredient of this product is azithromycin. Appearance: This product is a capsule with white or almost white crystalline powder as its contents. Indications: This product is used for pneumonia caused by Mycoplasma pneumoniae. Specification: 0.25 g/capsule. Dosage and Administration: Oral administration. For adults, 0.5 g once daily for 3 consecutive days. For children, the dosage is based on body weight at 10 mg/kg daily. Storage Conditions: Store in a tightly closed container in a dry place.

### 1.3 Experimental Animals

Balb/c mice, SPF grade, weighing 13-15 g, with a total of 100 mice, half male and half female.

1.4 Virus Strain: Mycoplasma Pneumoniae (MP), VR15531™.

### 1.5 Experimental Instruments

TABLE 16
Experimental Instruments Table

| Instrument Name | Model | Manufacturer | Purpose |
|---|---|---|---|
| Biosafety Cabinet | MSC1.2 | Thermo Fisher | Virus |
| Type A2 | | Scientific Inc. | Solution |
| | | | Preparation |
| Biosafety Cabinet | MSC1.8 | Thermo Fisher | Animal |
| Type A2 | | Scientific Inc. | Infection |
| Electronic | BSA3202S-CW | Sartorius | Weighing |
| Balance | | Scientific | Weight |
| | | Instruments Co., | |
| | | Ltd. | |
| Electronic | AL204 | Mettler-Toledo | Weighing |
| Balance | | Instruments Co., | Lung |
| | | Ltd. | Weight |
| Buxco Small | Buxco | DSI, USA | Drug |
| Animal Nose- | | | Administration |
| Only Exposure | | | in Mice |
| Tower | | | |

### 1.6 Experimental Reagents

TABLE 17
Experimental Reagents Table

| Reagent Name | Catalog Number | Manufacturer |
|---|---|---|
| Mouse IL-6 ELISA KIT | 202311 | Shanghai Yaji |
| | | Biological |
| | | Technology Co., Ltd. |
| Mouse IL-1β ELISA KIT | 202311 | Shanghai Yaji |
| | | Biological |
| | | Technology Co., Ltd. |
| Mouse TNF-α ELISA KIT | 202311 | Shanghai Yaji |
| | | Biological |
| | | Technology Co., Ltd. |
| Mouse CRP ELISA KIT | 033023231027 | Beyotime Biotech |
| | | Co., Ltd. |
| Mycoplasma Pneumoniae | Z-RD-0224-02-25 | Shanghai Zhijiang |
| and Chlamydia | | Biotechnology Co., |
| Pneumoniae Nucleic Acid | | Ltd. |
| Combined Detection Kit | | |

### 2. Experimental Methods and Results

### 2.1 Effect on Lung Index and Lung Index Inhibition Rate in Mice

Fifty BALB/C mice, weighing 14±1 g, were randomly divided into five groups: a normal control group, a model control group, an azithromycin control group, a high-dose Genipin-1-β-D-gentiobioside group (75 mg/ml) with 15-minute nebulization, and a low-dose Genipin-1-β-D-gentiobioside group (37.5 mg/ml) with 15-minute nebulization. Each group consisted of 10 mice, with an equal number of males and females. Except for the normal control group, all mice were lightly anesthetized with isoflurane and infected intranasally with Mycoplasma Pneumoniae (50 μl per mouse) for three consecutive days. On the day of infection, each treatment group received nebulized medication once daily for four consecutive days, while the normal control group and model control group received nebulized saline under the same conditions. On the fifth day, the mice were weighed, sacrificed by cervical dislocation, and their lung tissues were dissected, weighed, and used to calculate the lung index and lung index inhibition rate; measure the levels of inflammatory factors in the lung tissue; measure the serum CRP levels; and conduct pathological examinations of the lung tissue. The results were statistically analyzed using the t-test for intergroup comparisons.

$$Lung index = ( Lung wet weight ( g ) / Body weight ( g ) ) \times 100$$

$$Inhibition rate of lung index = ( Lung index of model control group - Lung index of drug administration group ) / ( Lung index of model control group - Lung index of normal control group ) \times 100 \%$$

TABLE 18
Therapeutic Effect of Genipin-1-β-D-Gentiobioside
on Mycoplasma Pneumoniae-Infected Mouse Pneumonia Model

| | | Inhalation | Number of | First Batch | | Second Batch | |
|---|---|---|---|---|---|---|---|
| | | Time | Animals | | Inhibition | | Inhibition |
| Group | Dosage | (min) | (n) | Lung Index | Rate | Lung Index | Rate |
| Normal Control | — | 15 | 10 | 0.71 ± 0.08 | — | 0.62 ± 0.03 | |
| Group | | | | | | | |
| Model Control | — | 15 | 10 | 0.89 ± 0.08^{##} | — | 0.76 ± 0.07^{##} | |
| Group | | | | | | | |
| Azithromycin | 42 | Administration | 10 | 0.77 ± 0.05** | 65.93 | 0.62 ± 0.09** | 98.72 |
| Group (mg/kg) | | by gavage | | | | | |
| | | Injection | 10 | | | 0.62 ± 0.06** | 98.26 |
| | | administration | | | | | |
| Genipin-1-β-D | 75 | 15 | 10 | 0.72 ± 0.05** | 93.06 | 0.67 ± 0.07** | 64.09 |
| gentiobioside | 37.5 | 15 | 10 | 0.76 ± 0.09** | 73.01 | 0.68 ± 0.08** | 56.34 |
| (mg/ml) | | | | | | | |

Note:
Compared with the normal control group, ^{##}p < 0.05; Compared with the model control group, **p < 0.01.

The results in Table 18 indicate that the lung index in the MP model control group was significantly increased, with a significant difference compared to the normal control group (p<0.01). After four days of nebulized administration of Genipin-1-β-D-gentiobioside, there were significant differences in the lung index between the high- and low-dose Genipin-1-β-D-gentiobioside groups and the model control group (p<0.01), and the experimental results were reproducible in two batches.

### 2.2 Detection of Inflammatory Factors in Mouse Lung Tissue (ELISA Method)

\textcircled{1} Sample Collection and Storage: Tissue Homogenate Samples: After weighing, lung tissues were collected from 8 mice in each group and stored at −80° C. The MP Fastprep-24 5G Rapid Sample Preparation System was used to homogenize the tissues, followed by centrifugation at −4° C. and 3000 r/min for 10 minutes using a low-temperature high-speed centrifuge. The supernatant was then aspirated, aliquoted, and stored at −80° C. for future use, avoiding repeated freeze-thaw cycles.

\textcircled{2} The microplate, which had been equilibrated to room temperature, was removed from the sealed bag. Different concentrations of standards were added to the corresponding wells, with 50 μL per well. For experimental samples, 10 μL of sample was added to each well, followed by 40 μL of diluent. Except for the blank wells, 100 μL of HRP (Horseradish Peroxidase) was added to each well. The reaction wells were sealed with sealing tape and incubated at 37° C. for 1 hour. The plate was then washed with wash buffer four times. After the final wash, the plate was inverted and all residual liquid was patted dry on absorbent paper. 50 μL of both Substrate A and Substrate B were added to each well. The reaction wells were then sealed with sealing tape and incubated at 37° C. for 15 minutes. After that, 50 μL of stop solution was added to each well, and the absorbance at 450 nm was measured using a microplate reader within 15 minutes. The results were then calculated.

TABLE 19
Therapeutic Effect of Genipin-1-β-D-Gentiobioside
on Mycoplasma Pneumoniae-Infected Mouse Pneumonia Model

| | | Number of | | | |
|---|---|---|---|---|---|
| | | Samples | Inflammatory Factor Levels in Lung Tissue (pg/mL) | | |
| Group | Dosage | (n) | IL-6 | IL-1β | TNF-α |
| Normal Control | — | 8 | 79.20 ± 32.91 | 106.53 ± 24.62 | 197.16 ± 58.41 |
| Group | | | | | |
| Model Control | — | 8 | 257.36 ± 25.76^{##} | 232.24 ± 35.96^{##} | 568.59 ± 38.02^{##} |
| Group | | | | | |
| Azithromycin | 42 (mg/kg) | 8 | 85.26 ± 25.59** | 144.98 ± 15.93** | 345.86 ± 53.85** |
| Control | | | | | |
| Genipin-1-β- | 75 (mg/ml) | 8 | 163.92 ± 8.54** | 148.31 ± 10.97** | 312.79 ± 81.52** |
| D-gentiobioside | 37.5 (mg/ml) | 8 | 150.92 ± 7.65** | 147.59 ± 41.87** | 248.53 ± 93.97** |

Note:
Compared with the normal control group,
^{##}p < 0.01; Compared with the model control group,
**p < 0.01.

The results in Table 19 indicate that the levels of inflammatory factors IL-6, IL-1β, and TNF-α in the lung tissue of the MP model control group were significantly increased, with a significant difference compared to the normal control group (p<0.01). After 4 days of inhalation administration of high and low doses of Genipin 1-β-D-gentiobioside, the levels of these inflammatory factors in the lungs of pneumonia mice were significantly reduced, with a significant difference compared to the model control group (p<0.01).

### 2.3 Detection of CRP in Mouse Serum (ELISA Method)

\textcircled{1}Sample Collection and Storage: Blood was collected from the orbital sinus of mice and allowed to stand for 2 hours. After centrifugation at 2000 g for 10 minutes, the supernatant was aspirated and stored at −80° C. for future use, avoiding repeated freeze-thaw cycles. For CRP detection, the samples were diluted 3000-fold with physiological saline.

\textcircled{2} The microplate, which had been equilibrated to room temperature, was removed from the sealed bag. Different concentrations of standards and samples were added to the corresponding wells, with 100 μL per well, and incubated at room temperature for 2 hours. The plate was then washed with wash buffer five times. Next, 100 μL of biotinylated antibody was added to each well and incubated at room temperature for 60 minutes. After washing the plate five times, 100 μL of horseradish peroxidase was added and incubated in the dark for 20 minutes. The plate was washed again five times, followed by the addition of chromogenic substrate and incubation for 20 minutes. Finally, 50 μL of stop solution was added to each well, and the absorbance at 450 nm was measured using a microplate reader within 15 minutes. The results were then calculated.

TABLE 20
Therapeutic Effect of Genipin-1-β-D-Gentiobioside on
Mycoplasma Pneumoniae (MP)-Infected Mouse Pneumonia Model

| | | Number of | CRP Levels in |
|---|---|---|---|
| | | Samples | Serum |
| Group | Dosage | (n) | (105 pg/ml) |
| Normal Control | — | 8 | 2.51 ± 0.47 |
| Group | | | |
| Model Control | — | 8 | 9.71 ± 2.71^{##} |
| Group | | | |
| Azithromycin | 42 (mg/kg) | 8 | 4.86 ± 1.21** |
| Control | | | |
| Genipin-1-β- | 75 (mg/ml) | 8 | 7.76 ± 1.72** |
| D-gentiobioside | 37.5 (mg/ml) | 8 | 6.65 ± 0.84** |

Note:
Compared with the normal control group,
^{##}p < 0.01; Compared with the model control group,
**p < 0.01.

The results in Table 20 indicate that the levels of CRP in the serum of mice in the MP model control group were significantly increased, with a significant difference compared to the normal control group (p<0.01). After 4 days of inhalation administration of high and low doses of Genipin 1-β-D-gentiobioside, the levels of CRP in the lungs of pneumonia mice were significantly reduced, with a significant difference compared to the model control group (p<0.01).

### 2.4 Pathological Changes in Mouse Lung Tissue (HE Staining Method)

Normal Control Group: The alveolar surface was smooth with no significant deformation or rupture. The epithelial cells of the bronchioles were arranged in order, and no edema, degeneration, or necrosis was observed. The lung interstitium showed no fibrous tissue proliferation or inflammatory cell infiltration, and the tissue structure was normal.

Model Control Group: Microscopically, numerous alveoli were observed to be congested, edematous, and exudative. Some alveolar walls were ruptured, leading to alveolar fusion and consolidation. The alveolar septa were mildly or moderately thickened, accompanied by a large number of inflammatory cell infiltrations, predominantly neutrophils, along with a few lymphocytes, macrophages, and plasma cells. The bronchial mucosal epithelial cells exhibited mild or slight edema and degeneration, and a small amount of necrotic and detached epithelium as well as inflammatory cells were visible in the lumen. There was a significant difference compared with the normal control group (P<0.01).

Genipin-1-β-D-gentiobioside (BD)-77 High-Dose Group: The alveoli of the mice showed focal mild or slight congestion and edema, with mild or slight thickening of the alveolar walls and a small amount of inflammatory cell infiltration predominantly composed of neutrophils. Some animals exhibited mild or slight edema of the bronchial epithelium, and the bronchial epithelial cells were arranged relatively regularly. The pathological changes were significantly reduced compared with the model group (P<0.1).

BD-77 Low-Dose Group: Microscopically, the alveolar walls of the mice were observed to be mildly or slightly thickened, accompanied by a small amount of inflammatory cell infiltration. The pathological changes were significantly reduced compared with the model group (P<0.1). The alveoli showed multifocal mild or slight congestion and edema, which were alleviated compared with the model group, but no statistical difference was observed. The bronchial mucosal epithelial cells exhibited mild to moderate edema and degeneration, and some bronchial epithelial cells were necrotic. There was no significant difference compared with the model group.

TABLE 21
Therapeutic effect of BD-77 on MP-infected mouse pneumonia model

| | | Degree of Histopathological Lesion | | | |
|---|---|---|---|---|---|
| | | | Interstitial | | Histological |
| | Number of | Alveolitis | Inflammatory Cell | Bronchial | Damage |
| Group | Samples | Lesion Degree | Infiltration | Damage | Degree |
| Normal Control | 10 | 5.50 | 5.50 | 7.50** | 0.00 ± 0.00 |
| Group | | | | | |
| Model Control | 10 | 52.10^{##} | 49.40^{##} | 48.80 | 2.20 ± 0.36^{##} |
| Group | | | | | |
| Azithromycin | 10 | 59.70 | 58.80 | 54.80 | 2.43 ± 0.32 |
| Group | | | | | |
| BD-77 High- | 10 | 30.40* | 30.60* | 26.60* | 1.50 ± 0.55* |
| Dose Group | | | | | |
| BD-77 Low- | 10 | 39.35 | 30.60* | 52.35 | 1.90 ± 0.39* |
| Dose Group | | | | | |

Pharmacological Experiment Embodiment 7: Therapeutic Effect of Intravenous Injection of Genipin-1-β-D-gentiobioside on Lipopolysaccharide (LPS)-Induced Pneumonia Model in Mice

### 1 Experimental Materials

1.1 Drug: Genipin-1-β-D-Gentiobioside preparation; Specification: 5 ml; 375 mg; Batch Number: 230608; Appearance: light yellow liquid; Storage: room temperature.

1.2 Animals: ICR mice, SPF grade, weighing 18˜20 g, 60 mice in total, half male and half female, provided by Beijing Vital River Laboratory Animal Technology Co., Ltd. (animal production license number: SCXK(Jing)2021-0006); housed in Animal Biosafety Level 2 (ABSL-2) biosafety rooms.

1.3 Reagents and Instruments: Lipopolysaccharide (LPS) produced by Sigma, batch number: lot #057M4013V Isoflurane, produced by JiangSu Hengfeng strong biological technology Co., Ltd., batch number: 20211202. Manufacturing date: Dec. 24, 2021, expiration date: Dec. 23, 2023. MC 1.8 type biosafety cabinet, Thermo company. BSA3202S-CW, BSA323S-CW electronic balances, Sartorius Instruments Co., Ltd. AL-204 type METTLER TOLEDO electronic balance, Mettler-Toledo Instruments (Shanghai) Co., Ltd.

### 2 Experimental Methods and Results:

### 2.1 Effect on Lung Index and Lung Index Inhibition Rate in Mice

Sixty healthy mice, weighing 18˜20 g, half male and half female, were selected and randomly grouped after stratification by weight; they were divided into a normal control group, a model control group, three Genipin-1-β-D-gentiobioside dose groups (150 mg/kg/d, 75 mg/kg/d, 37.5 mg/kg/d), and a positive control drug Sivelestat Sodium 50 mg/kg/d group, with 10 mice in each group, half male and half female. After grouping, each treatment group was administered intravenously at 20 ml/kg once daily for 2 consecutive days, while the normal control group and model control group were injected with physiological saline. One hour after the second dose, except for the normal control group, the other groups were lightly anesthetized with isoflurane, and a 20 mg/ml LPS physiological saline solution was administered intranasally to the mice at 0.05 ml/mouse to induce the pneumonia model. Six hours after infection, the animals were sacrificed, dissected, and their lungs were removed and weighed to calculate the lung index (lung index=lung weight/body weight×100%).

TABLE 22
Effect of Genipin-1-β-D-gentiobioside
on LPS-Induced Pneumonia Model in Mice

| | | | Lung Index | |
|---|---|---|---|---|
| | Dosage | Number of | (g/100 g | Inhibition |
| Group | (mg/kg) | Animals (10) | body weight) | Rate |
| Normal | 20 | 10 | 0.84 ± 0.07 | — |
| Control | | | | |
| Group | | | | |
| Model | 20 | 10 | 0.98 ± 0.08^{##} | — |
| Control | | | | |
| Group | | | | |
| Sivelestat | 50 | 10 | 0.86 ± 0.07** | 85.50 |
| Sodium | | | | |
| Genipin-1-β- | 150 | 10 | 0.87 ± 0.01# | 77.10 |
| D-gentiobioside | 75 | 10 | 0.85 ± 0.14# | 94.65 |
| | 37.5 | 10 | 0.91 ± 0.09 | 48.85 |

Note:
Compared with the normal control group,
^{##}p < 0.01; Compared with the model control group,
**p < 0.01.

The results in Table 22 indicate that the lung index of mice in the model control group was significantly increased 6 hours after LPS inhalation, with a significant difference compared to the normal control group (p<0.01). Genipin-1-β-D-gentiobioside, at doses of 150 mg/kg and 75 mg/kg, showed a significant decrease in lung index in the high and medium dose groups, with a significant difference compared to the model control group (p<0.01, p<0.05, respectively). These results indicate that Genipin-1-β-D-gentiobioside at doses of 150 mg/kg and 75 mg/kg has a protective effect against LPS-induced pneumonia in mice.

### 2.2 Detection of Inflammatory Factors in Mouse Lung Tissue (ELISA Method)

\textcircled{1} Sample Collection and Storage: Tissue homogenate samples: The lung tissues of mice were collected and weighed, including those from mice numbered 1, 2, 3, 6, 7, and 8 in each group. The tissues were stored at −4° C. An ultrasonic cell disruptor was used to homogenize the tissues, followed by centrifugation at −4° C. and 1000 r/min for 10 minutes using a low-temperature high-speed centrifuge. The supernatant was then collected, aliquoted, and stored at −80° C. for future use, avoiding repeated freeze-thaw cycles.

\textcircled{2} The microwell plate, which had been equilibrated to room temperature, was removed from the sealed bag. Different concentrations of standards, experimental samples, or quality control samples were added to the corresponding wells, with 100 μL per well. The reaction wells were sealed with sealing tape and incubated at room temperature for 2 hours. The plate was then washed with wash buffer, repeating the operation four times. After the final wash, the plate was inverted and all residual liquid was patted dry on absorbent paper. Next, 100 μL of enzyme-labeled detection antibody was added to each microwell. The reaction wells were sealed again with sealing tape and incubated at room temperature for another 2 hours. The washing step was repeated as described above. Then, 100 μL of substrate solution was added to each microwell and incubated at room temperature for 30 minutes, protected from light. Within 30 minutes after adding 100 μL of stop solution to each microwell, the absorbance at 450 nm was measured using a microplate reader. The results were then calculated.

TABLE 23
Effect of Genipin-1-β-D-gentiobioside on LPS-Induced Pneumonia Model in Mice

| | | Number of | | | |
|---|---|---|---|---|---|
| | Dosage | Animals | Inflammatory Factor Levels in Lung Tissue (pg/mL) | | |
| Group | (mg/ml) | (n) | IL-6 | IL-10 | TNF-α |
| Normal | — | 10 | 15.53 ± 1.82 | 36.25 ± 6.67 | 90.00 ± 7.35 |
| Control | | | | | |
| Group | | | | | |
| Model | — | 10 | 21.60 ± 1.19^{##} | 43.00 ± 4.98# | 133.96 ± 8.96^{##} |
| Control | | | | | |
| Group | | | | | |
| Sivelestat | 50 | 10 | 15.79 ± 2.08** | 36.11 ± 4.87** | 98.88 ± 11.21** |
| Sodium | | | | | |
| Genipin-1-β- | 150 | 10 | 16.63 ± 2.19** | 36.00 ± 5.14** | 100.25 ± 12.41** |
| D-gentiobioside | 75 | 10 | 17.72 ± 2.15** | 35.75 ± 6.74* | 119.71 ± 19.76* |
| | 37.5 | 10 | 16.53 ± 1.91** | 39.00 ± 8.37 | 106.13 ± 15.70** |

Note:
Compared with the normal control group,
^{##}p < 0.01; Compared with the model control group,
*p < 0.05,
**p < 0.01.

The results in Table 23 indicate that the levels of IL-6, IL-10, and TNF-α in the lung tissue of mice in the model control group were significantly increased 6 hours after LPS inhalation, with significant differences compared to the normal control group (p<0.01, p<0.05, respectively). Genipin-1-β-D-gentiobioside administration groups showed a significant decrease in the levels of IL-6, IL-10, and TNF-α, with significant differences compared to the model control group (p<0.01, p<0.05, respectively). These results suggest that Genipin-1-β-D-gentiobioside at doses of 150 mg/kg and 75 mg/kg has a protective effect against LPS-induced pneumonia in mice.

What is claimed is:

1. A use of a Genipin-1-β-D-gentiobioside in preparation of a drug, wherein the Genipin-1-β-D-gentiobioside is used in preparation of a drug for treating respiratory injury caused by a coronavirus, respiratory syncytial virus or Mycoplasma pneumoniae infection.

2. The use of the Genipin-1-β-D-gentiobioside in preparation of the drug according to claim 1, wherein the Genipin-1-β-D-gentiobioside is used in preparation of a drug for treating pneumonia inflammatory injury caused by the coronavirus, respiratory syncytial virus or Mycoplasma pneumoniae infection.

3. The use of the Genipin-1-β-D-gentiobioside in preparation of the drug according to claim 1, wherein the Genipin-1-β-D-gentiobioside is used in preparation of a drug for treating coronavirus, respiratory syncytial virus or Mycoplasma pneumoniae infections, which further exhibits a protective effect against mortality.

4. The use of the Genipin-1-β-D-gentiobioside in preparation of the drug according to claim 1, wherein the coronavirus comprises any one of Severe Acute Respiratory Syndrome Coronavirus 2 (SARS-CoV-2), Human Coronavirus 229E (HCoV-229E) and Human Coronavirus OC43 (HCoV-OC43).

5. The use of the Genipin-1-β-D-gentiobioside in preparation of the drug according to claim 1, wherein administration routes of the Genipin-1-β-D-gentiobioside comprise any one of inhalation, oral administration, injection, sublingual administration, spraying or rectal administration; and dosage forms of the drug comprise any one of inhalation, oral, injection, spray, film and suppository.

6. A nebulized inhalation solution containing a Genipin-1-β-D-gentiobioside, achieving the use according to claim 1, wherein the nebulized inhalation solution comprises following ingredients: the Genipin-1-β-D-gentiobioside, a pH regulator that adjusts pH to 4.5˜7.0, an osmotic pressure regulator that accounts for 0˜0.9% of a weight of the Genipin-1-β-D-gentiobioside and a solvent.

7. The nebulized inhalation solution containing the Genipin-1-β-D-gentiobioside according to claim 6, wherein a preparation method of the nebulized inhalation solution comprises following steps:
(1) measuring 40%˜90% (v/v) of total solution volume required for preparation using water for injection to obtain a first solution;
(2) adding the osmotic pressure regulator into the first solution at a temperature of 20˜80° C., and stirring uniformly to obtain a second solution;
(3) adding the pH regulator and the Genipin-1-β-D-gentiobioside into the second solution, and adjusting a pH of the second solution to a target value to obtain a third solution; and
(4) adding solvent into the third solution to reach total solution volume required for preparation, and stirring uniformly to obtain the nebulized inhalation solution containing the Genipin-1-β-D-gentiobioside.

8. An injection containing a Genipin-1-β-D-gentiobioside, achieving the use according to claim 1, wherein the injection comprises following ingredients: the Genipin-1-β-D-gentiobioside, a pH regulator that adjusts pH to 4.5-7.0, an osmotic pressure regulator that adjusts the osmolarity to isotonicity and an injection solvent.

9. The injection containing the Genipin-1-β-D-gentiobioside according to claim 8, wherein a preparation method of the injection comprises following steps:
(1) measuring 40%˜90% (v/v) of total solution volume required for preparation using water for injection to obtain a first solution;
(2) adding the osmotic pressure regulator into the first solution at a temperature of 20˜80° C., and stirring uniformly to obtain a second solution;
(3) adding the pH regulator and the Genipin-1-β-D-gentiobioside into the second solution, and adjusting a pH of the second solution to a target value to obtain a third solution; and
(4) adding solvent into the third solution to reach total solution volume required for preparation, and stirring uniformly to obtain the injection containing the Genipin-1-β-D-gentiobioside.

10. A preparation method of a Genipin-1-β-D-gentiobioside, used for extracting the Genipin-1-β-D-gentiobioside in any one of the uses according to claim 1, wherein the method comprises following operation steps:
a, taking Gardeniae Fructus, performing water extraction to obtain an extract, concentrating the extract under reduced pressure to achieve a crude drug content of 0.03˜0.2 g/ml in the extract;
b, loading the extract onto a macroporous resin column, eluting first with 1˜5 times column volumes of deionized water, then eluting with 1˜5 times column volumes of 10˜20% ethanol, collecting ethanol eluate, recovering ethanol under reduced pressure, concentrating to a volume that is 0.1 times the original volume of the crude drug extract, adding ethanol to achieve a 90% ethanol concentration, letting the solution stand, allowing precipitation, filtering, passing supernatant obtained from ethanol precipitation through a neutral alumina column, eluting sequentially with 1˜8 times column volumes of 50˜90% ethanol, collecting ethanol eluate obtained with 50˜60% ethanol, recovering ethanol under reduced pressure, and drying to obtain a crude product; and
c. refining the crude product by hot ethanol dissolution and recrystallization for 2˜3 times to obtain a refined product, drying the refined product, and removing ethanol to obtain high-purity Genipin-1-β-D-gentiobioside.

11. The preparation method of the Genipin-1-β-D-gentiobioside according to claim 10, wherein in step a, the specific steps for water extraction are: crushing Gardeniae Fructus, boiling crused Gardeniae Fructus with 12 times, 10 times and 10 times the amount of water respectively for 1˜1.5 hours each time; and in step b, a wet volume ratio of macroporous resin to a weight of Gardeniae Fructus is 3:2˜3 ml/g, and a weight ratio of neutral alumina to the weight of Gardeniae Fructus is 1:3˜3.5.

12. The preparation method of the Genipin-1-β-D-gentiobioside according to claim 10, wherein step b specifically involves loading Gardeniae Fructus extract onto an NKA-9 macroporous resin column, eluting first with 2 times column volumes of deionized water, collecting sample solution and water eluate, combining the sample solution and the water eluate, and then loading the combined solution onto an X-5 macroporous resin column, eluting first with 1 times column volume of deionized water, then eluting with 5 times column volumes of 10% ethanol, collecting ethanol eluate, recovering ethanol under reduced pressure, concentrating to a relative density of 1.08˜1.15 at 60° C., adding ethanol to achieve a 90% ethanol concentration, letting the solution stand, allowing precipitation, filtering, passing supernatant obtained from ethanol precipitation through the neutral alumina column, eluting sequentially with 6 times column volumes of 90% ethanol and 4 times column volumes of 60% ethanol, collecting eluate obtained with 60% ethanol, recovering ethanol under reduced pressure, and drying to obtain the crude product.

13. The preparation method of the Genipin-1-β-D-gentiobioside according to claim 10, wherein in step c, the specific steps for recrystallization after hot ethanol dissolution of the crude product are: adding the crude product to 0.5˜1 times the amount of anhydrous ethanol, heating and refluxing to dissolve the crude product, filtering while hot, letting the solution stand, allowing crystallization, and filtering by suction to obtain the refined product.
